(19) United States
(12) Patent Application Publication
(10) Pub. No.: US 2026/0028599 A1
(43) Pub. Date: Jan. 29, 2026

(54) EPIGENOME EDITORS FOR MODULATING HIV TRANSCRIPTION

(71) Applicants: Francisco J. Zapatero Belinchon, San Francisco, CA (US); Melanie Ott, Mill Valley, CA (US); Ulrike C Lange, Hamburg (DE); Martin Volker Hamann, Hamburg (DE)

(72) Inventors: Francisco J. Zapatero Belinchon, San Francisco, CA (US); Melanie Ott, Mill Valley, CA (US); Ulrike C Lange, Hamburg (DE); Martin Volker Hamann, Hamburg (DE)

(21) Appl. No.: 19/281,000

(22) Filed: Jul. 25, 2025

Related U.S. Application Data
(60) Provisional application No. 63/675,718, filed on Jul. 25, 2024.

(51) Int. Cl. C12N 9/10; A61K 38/00; C07K 14/47; C12N 9/12

(52) U.S. Cl. CPC C12N 9/1007; C07K 14/4703; C12N 9/1241; C12Y 201/01037; A61K 38/00; C07K 2319/00

(57) ABSTRACT

BrecOFF is an epigenome editor that specifically targets the HIV promoter, long tandem repeat (LTR), to deposit DNA and histone repressive markers and thereby silencing its transcription and reactivation. BrecOFF comprises of 3 modules (FIG. 1a). The DNMT3A and cofactor DNMT3L are the de novo DNA methylation machinery that marks DNA with repressive methylation marks. The Krüppel associated box (KRAB) domain is a transcriptional repression domain by recruit of TRIM28 repression complex and addition of repressive epigenetic marks on histones. Finally, dBrec1 is a catalytically inactive version of Brec1, a Cre-based directly evolved recombinase that specifically recognizes the R region of the HIV LTR without off-target effects

### PRIORITY

This application claims the benefit of the filing date of U.S. provisional application No. 63/675,718, filed Jun. 25, 2024, the disclosure of which is incorporated by reference herein.

### GOVERNMENT FUNDING

This invention was made with government support under AI164599 awarded by the National Institutes of Health. The government has certain rights in the invention.

### INCORPORATION BY REFERENCE OF SEQUENCE LISTING

This application contains a Sequence Listing which has been submitted electronically in ST26 format and hereby incorporated by reference in its entirety. Said ST26 file, created on Jul. 24, 2025, is named 3730233US1.xml and is 26,285 bytes in size.

### BACKGROUND OF THE INVENTION

About 39 million people are living with HIV (PLWH) worldwide as of the end of 2022. With the advent of antiretroviral therapies (ARTs), HIV has become manageable and PLWH can live a relatively healthy life without progression to acquired immunodeficiency syndrome (AIDS). However, once acquired, HIV is lifelong and not curable even under ART treatment. In addition, ART treatments have side-effects, and its disruption leads to viral rebound and disease progression, making ART treatment lifelong.

### SUMMARY OF THE INVENTION

Aspects provide a fusion protein comprising a DNA methyltransferase domain, a catalytically inactive Brec1 (dBrec1; in which Brec1 is mutated to generate dBrec1; wherein the mutation is at residue 324 of Brec; wherein tyrosine at residue 324 in Brec is mutated to a phenylalanine) and a Kruppel-associated box domain. In some aspects, the DNA methyltransferase domain comprises a Dnmt3A domain. In some aspects, the Dnmt3A domain is linked to a Dnmt3L domain (Dnmt3A-3L domain). Some aspects provide from N-terminus to C-terminus, a DNA methyltransferase domain, a catalytically inactive Brec1, and a Kruppel-associated box domain. Some aspects further provide an epitope tag, a fluorescent protein tag, a nuclear localization signal peptide, or a combination of two or more thereof. Some aspects further provide one or more linkers, such as XTEN linkers.

Some aspects provide a cell comprising the fusion protein described herein, such a eukaryotic cell, a mammalian cell, or a stem cell.

Aspects provide a method of silencing virus, such as HIV, in a cell, the method comprising delivering a polynucleotide encoding the fusion protein described herein to a cell containing HIV thereby silencing HIV. Aspects provide a method of treating HIV in a subject in need thereof, the method comprising delivering to the subject an effective amount of a polynucleotide encoding the fusion protein described herein thereby treating HIV, such as delivery in a nanoparticle or virus like particle (VLP).

These and other embodiments and aspects of the disclosure are described in detail herein.

### BRIEF DESCRIPTION OF THE FIGURES

FIGS. 1A, 1A1, 1A2, 1C and 1D. A. BrecOFF construct and alternative modules. The core BrecOFF construct is represented in the inner ring. Alternative modules are shown as ORFs outside the ring. 1A1 and 1A2 demonstrate “Block-Lock-Stop” Approach. B. BrecOFF modules and main advantages. C. BrecOFF represses episomal HIV LTR transcription by promoter occupancy. HEK293T cells were co-transfected with a HIV LTR-FLc plasmids together with dBrec1 or BrecOFF. Two days post-transfection cells were lysed and FLuc activity measured by standard procedures. HIV-LTR activity was normalized to HIV LTR-FLuc plasmid alone. D. BrecOFF transient expression durably silences basal HIV LTR transcription. TZM-bl (expressing FLuc under the HIV LTR promoter) were transfected with dBrec1-GFP or BrecOFF using FuGene HD. Next day, expressing cells were sorted by their fluorescence expression (GFP for dBrec1 and mTagBFP2 for BrecOFF). Sorted cells were cultured and HIV LTR activity was assessed at the indicated days. Unsorted TZM-bls were used as control for maximum HIV LTR activity. Altogether, this data indicates that both dBrec1 and BrecOFF bind specifically to the HIV LTR but only BrecOFF leaves epigenome marks that inhibits HIV transcription for at least 1 month after expression.

FIG. 2. pMAXBrecOFF (BrecOFF) variants repress HIV LTR activity in a dose-dependent manner. BrecOFF variants were transfected together with a vector expressing HIV LTR-Fluc similarly as for FIG. 1C. GFP was used as a control for transfection-driven inhibition.

FIG. 3. BrecOFF HIV silencing depends on KRAB domain repression potency. BrecOFF and KRAB domain variants ZIM2 and ZIM3 were co-transfected with an HIV LTR-FLuc plasmid as described in FIG. 1C. GFP alone was used as transfection control. HIV transcription inhibition is shown as the percentage of LTR activity normalized to LTR-FLuc alone. These data suggest the role of the KRAB domain for HIV transcription repression as well as the plasticity of BrecOFF for new modules that can increase repression potency.

FIG. 4. BrecOFF diminishes HIV reactivation in T cell latency models. J-Lat 11.1 and J-Lat 5A8 were stable transduced with inducible dBrec1 or BrecOFF (idBrec1, iBrecOFF). Generated cell lines were treated with 500 ng/mL Doxycycline (Dox) to induce expression of proteins. 2 days after induction, integrated HIV was reactivated with 5 or 10 ng/mL TNFa for 18 h and measured with flow cytometry. HIV reactivation was assessed as percentage of GFP+ cells in cells expressing dBrec1 or BrecOFF. These data indicate that BrecOFF induces episomal marks that block HIV reactivation in T cells.

FIG. 5. Generation and expression of BrecOFF in virus-like particles (VLPs) based on Banskota et. al (Banskota S, et al. Engineered virus-like particles for efficient in vivo delivery of therapeutic proteins. Cell. 2022). Diagram shows generated constructs for expression of dBrec1 and BrecOFF within VLPs as well as the workflow to generate VLPs.

FIG. 6. dBrec1 and BrecOFF are packaged into VLPs. Purified VLPs were prepared for Western Blotting. A. HA-tag blot. Uncleaved gag_dBrec1 is at 141 kDa and gag_BrecOFF at 219 kDa. B. VSV-G blot showing the protein at ˜65 kDa. In addition, VLPs packaged MLV gag as measured by a MuLV core Antigen ELISA.

FIG. 7. BrecOFF VLPs repress HIV LTR transcription. VLPs were generated in 293T using different gag-pro-pol-Gag-epigenome editor ratios. Generated VLPs were used to inoculate TZM-bl cells for 48 hours prior to cell lysis and luciferase measurements. Luciferase activity was normalized to untreated cells. Both dBrec1 and BrecOFF inhibit HIV transcription in a dose-dependent manner, indicating that both proteins are released from Gag and localized to the nucleus where they bind to the HIV LTR and block transcription.

FIG. 8. BrecOFF VLPs Have Additional Silencing on HIV LTR.

### DETAILED DESCRIPTION OF THE INVENTION

A new approach for an HIV cure, or cure/treatment for other viruses, is to pursue a “functional cure.” This type of cure would entail absence of viral replication and rebound even in the absence of ART treatment, as well as no risk of transmission and pathology. Previous work with HIV and recent findings with endogenous retrovirus (ERVs), which make up to 8% of our genetic material, has shown that retroviruses can go into “deep latency” by epigenetic repression. In this state, retroviruses do not reactivate and are transcriptionally silent, or in other words, non-productive.

Provided herein are compositions and methods to induce a “deep latency” state in, for example, HIV infected cells so as to treat patients with a functional HIV cure, by forcing HIV in a durable silent dormancy. This invention is complementary with other silencing promoting agents (SPAs) within the “block-lock-stop” approach of HIV cure.

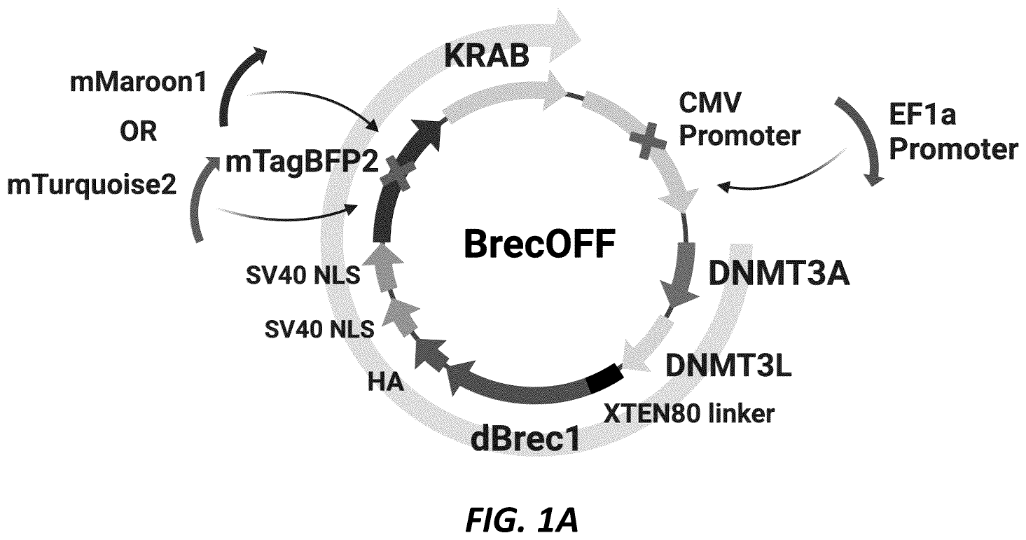

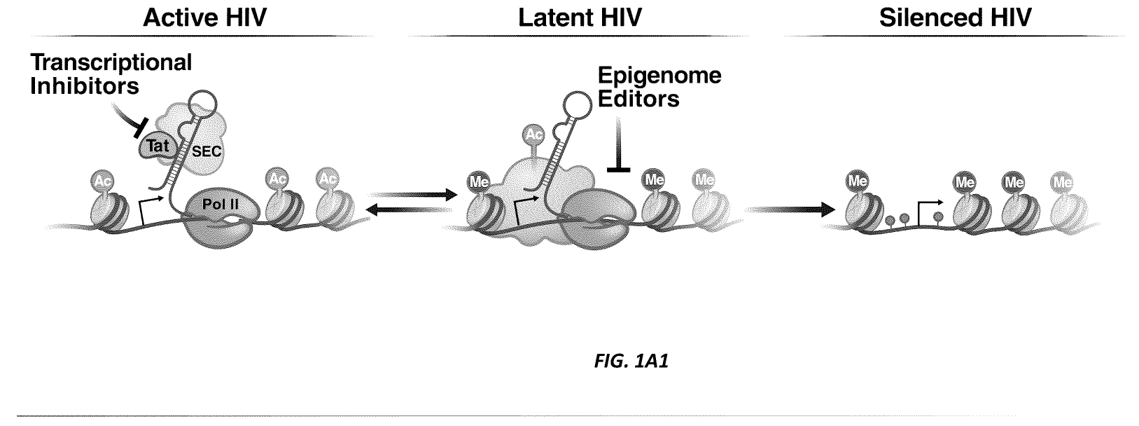

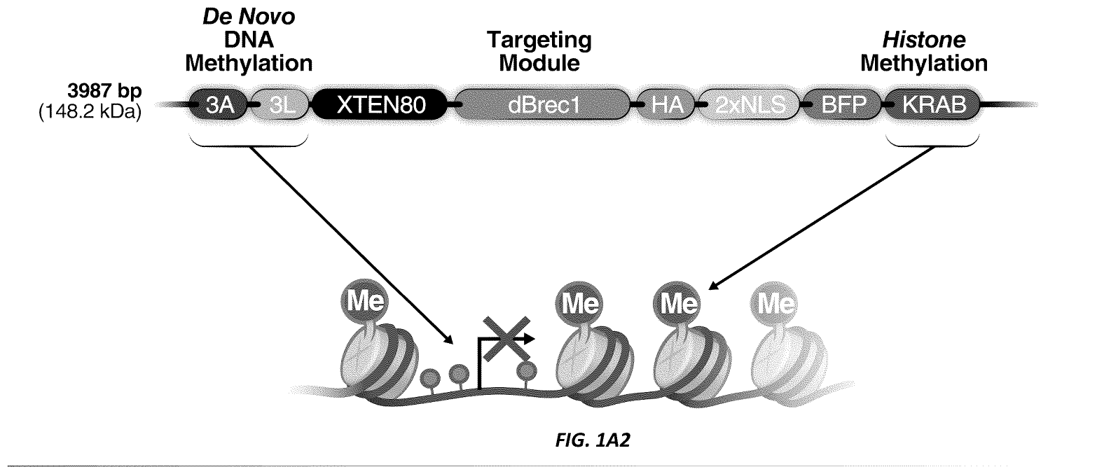

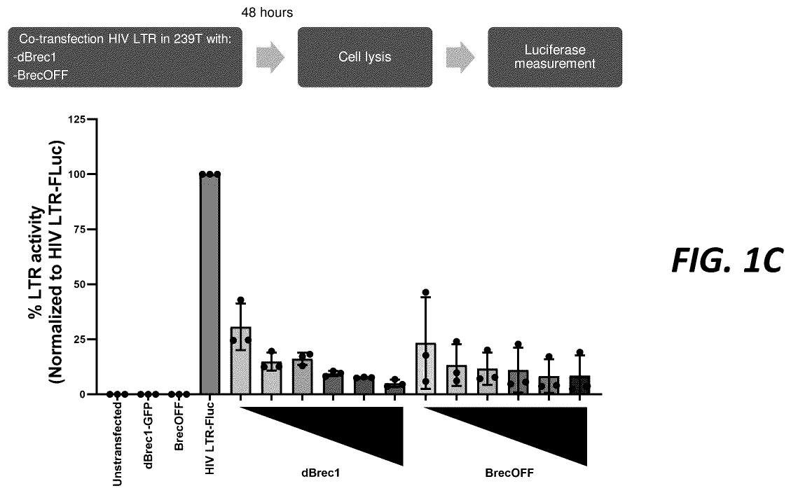

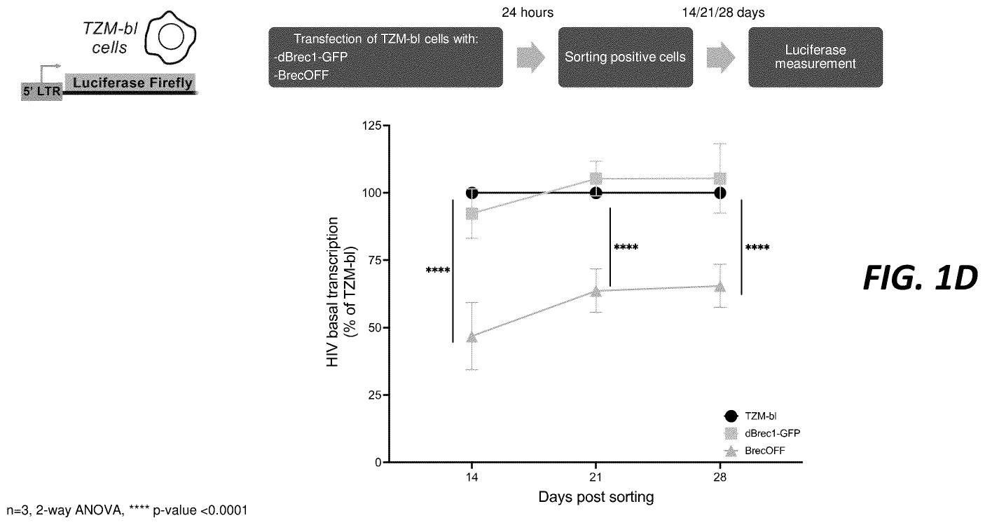

BrecOFF is an epigenome editor that specifically targets the HIV promoter, long tandem repeat (LTR), to deposit DNA and histone repressive markers and thereby silencing its transcription and reactivation. BrecOFF consist of 3 modules (FIG. 1A). The DNMT3A and cofactor DNMT3L are the de novo DNA methylation machinery that marks DNA with repressive methylation marks. The Krüppel associated box (KRAB) domain is a transcriptional repression domain by recruit of TRIM28 repression complex and addition of repressive epigenetic marks on histones. Finally, dBrec1 is a catalytically inactive version of Brec1, a Cre-based directly evolved recombinase that specifically recognizes the R region of the HIV LTR without off-target effects (Karpinski et al. Nat Biotechnol. 2016 April; 34(4):401-9. doi: 10.1038/nbt.3467. Epub 2016 Feb. 22.). BrecOFF was constructed by replacing CRISPRoff's Cas9 (Nunez et al. Cell. 2021 Apr. 29; 184(9):2503-2519.e17. doi: 10.1016/j.cell.2021.03.025. Epub 2021 Apr. 9.) with dBrec1. Alternative version of modules such as fluorescent reporters or other KRAB domains can also be generated.

One advantage of BrecOFF compared to other epigenome editors is its specificity and size. The replacement of dCas9 with dBrec1 not only specifically directs the protein to a highly conserved region of HIV (over 90% sequence conservation based on Los Alamos HIV database) but also reduces its size by more than half (7041 base pair and 267.4 KDa for CRISPRoff vs. 3987 base pair and 148.2 KDa for BrecOFF), facilitating its delivery as genetic material or protein into the desired target cell or tissue. Furthermore, BrecOFF does not need guide RNA for genetic targeting and is not immunogenic.

BrecOFF expression construct can be made by fusing the de novo methylation machinery (DNMT3A and 3L), the KRAB transcriptional repressor domain, and the HIV LTR-specific dBrec1. Repression studies were conducted in vitro (transcriptional silencing and reactivation blockage) as well as ex vivo (primary T cell models) and can be carried out in vivo (mouse studies). Finally, patients can be treated with BrecOFF as a gene therapy agent alone or together with other SPAs.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2^{nd} ed. 1994); The Cambridge Dictionary of Science and Technology (Walker ed., 1988); The Glossary of Genetics, 5th Ed., Rieger et al. (eds.), Springer Verlag (1991); and Hale & Marham, The Harper Collins Dictionary of Biology (1991). As used herein, the following terms have the meanings ascribed to them unless specified otherwise.

The use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

For specific proteins described herein (e.g., KRAB, dBrec1, Dnmt3A, Dnmt3L), the named protein includes any of the protein's naturally occurring forms, or variants or homologs that maintain the protein activity (e.g., within at least 50%, 80%, 90%, 95%, 96%, 97%, 98%, 99% or 100% activity compared to the native protein). In aspects, variants or homologs have at least 50%, 55%, 60%, 65%, 70%, 75%, 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form In aspects, the protein is the protein as identified by its NCBI sequence reference. In aspects, the protein is the protein as identified by its NCBI sequence reference or functional fragment or homolog thereof.

Brec1 is a recombinase that site-specifically recognizes a 34-bp sequence present (5′-AACCCACTGCTTAAGCCTCAATAAAGCTTGCCTT-3′; SEQ ID NO: 29) in the long terminal repeats (LTRs) of the majority of the clinically relevant HIV-1 strains and subtypes (Karpinski et al. Nat Biotechnol. 2016 April; 34(4):401-9. doi: 10.1038/nbt.3467. Epub 2016 Feb. 22). Brec1 efficiently, precisely and safely removes the integrated provirus from infected cells and is efficacious on clinical HIV-1 isolates in vitro and in vivo (Id).

Brec includes the sequence set forth by SEQ ID NO: 1:

SILLTLHQSLSALLVDATSDEARKNLMDVLRDRQAFSERTWKVLLSVCR
IWAAWCKLNNRKWFPAEPEDVRDYLLHLQARGLAVNIILQHLAQLNMLH
RRFGLPRPGDSDAVSLVMRRIRRENVDAGERTKQALAFERTDFDQVRAL
MENSERGQDIRILALLGVAYNTLLRVSEIARIRIKDISRIDGGRMLIHI
SRTKTLVSTAGVEKALSLGVTKLVERWISVSGVASDPNNYLFCQVRING
VAVPSATSRLSTDVLRKIFEAAHRLIYGAKDGSGQRYLAWSGHSARVGA
ARDMARAGVSIAEIMQAGGWTTVESVMNYIRNLDSETGAMVRLLEDGD*

An example of a Brec1 nucleic acid sequence:

(SEQ ID NO: 30)
ATGTCCATCCTGCTGACCCTGCACCAGAGCCTGTCTGCCCTGCTGGTGG
ATGCCACCTCCGACGAGGCCCGGAAGAACCTGATGGACGTGCTGAGAGA
CAGACAGGCCTTCAGCGAGCGGACCTGGAAGGTGCTGCTGAGCGTGTGT
AGAACCTGGGCCGCCTGGTGCAAGCTGAACAACCGGAAGTGGTTCCCCG
CCGAGCCTGAGGATGTGCGGGACTACCTGCTGCATCTGCAAGCCAGAGG
CCTGGCCGTGAACACCATCCTGCAGCATCTGGCCCAGCTGAACATGCTG
CACAGAAGATTCGGCCTGCCCAGACCCGGCGATAGCGACGCTGTGTCTC
TCGTGATGCGGCGGATCCGGCGCGAGAATGTGGATGCCGGCGAGAGAAC
AAAGCAGGCCCTGGCCTTCGAGAGAACCGACTTCGATCAAGTGCGGGCC
CTGATGGAAAACAGCGAGAGAGGCCAGGACATCCGGACCCTGGCTCTGC
TGGGCGTGGCCTACAATACCCTGCTGCGGGTGTCCGAGATCGCCCGGAT
CAGAATCAAGGACATCAGCCGGACCGACGGCGGCAGAATGCTGATCCAC
ATCTCCCGGACCAAGACCCTGGTGTCCACCGCTGGCGTGGAAAAGGCCC
TGTCTCTGGGCGTGACCAAGCTGGTGGAACGGTGGATCTCCGTGTCTGG
CGTGGCCAGCGACCCCAACAACTACCTGTTCTGCCAAGTGCGGATCAAC
GGCGTGGCCGTGCCTTCCGCTACAAGCAGACTGAGCACCGACGTGCTGC
GCAAGATCTTCGAGGCCGCCCACAGACTGATCTACGGCGCCAAGGATGG
CAGCGGCCAGAGATACCTGGCTTGGAGCGGACACAGCGCCAGAGTGGGA
GCCGCTAGAGATATGGCCAGAGCCGGCGTGTCCATTGCCGAGATCATGC
AGGCTGGCGGCTGGACCACAGTGGAAAGCGTGATGAACTACATCCGCAA
CCTGGACAGCGAAACCGGCGCCATGGTGCGCCTGCTGGAAGAT

dBrec1 is a catalytically inactive version of Brec1, a Cre-based directly evolved recombinase that specifically recognizes the R region of the HIV LTR without off-target effects (Id). A mutation Brec destroys its recombinase activity but allows it to maintain its DNA binding activity. For example, tyrosine at residue 323 in Brec was mutated to a phenylalanine.

(SEQ ID NO: 2)
SILLTLHQSLSALLVDATSDEARKNLMDVLRDRQAFSERTWKVLLSVCR
TWAAWCKLNNRKWFPAEPEDVRDYLLHLQARGLAVNTILQHLAQLNMLH
RRFGLPRPGDSDAVSLVMRRIRRENVDAGERTKQALAFERTDFDQVRAL
MENSERGQDIRTLALLGVAYNTLLRVSEIARIRIKDISRTDGGRMLIHI
SRTKTLVSTAGVEKALSLGVTKLVERWISVSGVASDPNNYLFCQVRING
VAVPSATSRLSTDVLRKIFEAAHRLIYGAKDGSGQRYLAWSGHSARVGA
ARDMARAGVSIAEIMQAGGWTTVESVMNFIRNLDSETGAMVRLLEDGD*

This mutation in CRE destroys its recombinase activity but allows it to maintain its DNA binding activity (dBrec1; SEQ ID NO: 2). In aspects, the dBrec1 includes the sequence set forth by SEQ ID NO: 2. In aspects, the dBrec1 is the sequence of SEQ ID NO: 2. In aspects, the dBrec1 includes an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO: 2. In aspects, the dBrec1 includes an amino acid sequence that has at least 75% sequence identity to SEQ ID NO: 2. In aspects, the dBrec1 includes an amino acid sequence that has at least 80% sequence identity to SEQ ID NO: 2. In aspects, the dBrec1 includes an amino acid sequence that has at least 85% sequence identity to SEQ ID NO: 2. In aspects, the dBrec1 includes an amino acid sequence that has at least 90% sequence identity to SEQ ID NO: 2. In aspects, the dBrec1 includes an amino acid sequence that has at least 95% sequence identity to SEQ ID NO: 2.

The term “Kruppel associated box domain” or “KRAB domain” as provided herein refers to a category of transcriptional repression domains present in approximately 400 human zinc finger protein-based transcription factors. KRAB domains typically include about 45 to about 75 amino acid residues. A description of KRAB domains, including their function and use, may be found, for example, in Ecco, G., Imbeault, M., Trono, D., KRAB zinc finger proteins, Development 144, 2017; Lambert et al. The human transcription factors, Cell 172, 2018; Gilbert et al., Cell (2013); and Gilbert et al., Cell (2014). In aspects, the KRAB domain is a KRAB domain of Kox 1. In aspects, the KRAB domain includes the sequence set forth by SEQ ID NO: 3.

DAKSLTAWSRTLVTFKDVFVDFTREEWKLLDTAQQIVYRNVMLENYKNL
VSLGYQLTKPDVILRLEKGEEP
(SEQ ID NO: 3; KRAB; from Gilbert et al., Cell,
2013, 2014)

In aspects, the KRAB domain is the sequence of SEQ ID NO: 3. In aspects, the KRAB domain includes an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO: 3. In aspects, the KRAB domain includes an amino acid sequence that has at least 75% sequence identity to SEQ ID NO: 3. In aspects, the KRAB domain includes an amino acid sequence that has at least 80% sequence identity to SEQ ID NO: 3. In aspects, the KRAB domain includes an amino acid sequence that has at least 85% sequence identity to SEQ ID NO: 3. In aspects, the KRAB domain includes an amino acid sequence that has at least 90% sequence identity to SEQ ID NO: 3. In aspects, the KRAB domain includes an amino acid sequence that has at least 95% sequence identity to SEQ ID NO: 3. In embodiments, the KRAB domain is a ZIM3 KRAB domain or an amino acid sequence having 85%, 90%, or 95% sequence identity thereto. In embodiments, the KRAB domain is KOX1 or an amino acid sequence having 85%, 90%, or 95% sequence identity thereto. Gilbert et al, Cell, 159:647-661 (2014); Alerasool et al, Nature Methods, (2020).

The term “DNA methyltransferase” as provided herein refers to an enzyme that catalyzes the transfer of a methyl group to DNA. Non-limiting examples of DNA methyltransferases include Dnmtl, Dnmt3A, and Dnmt3B. In aspects, the DNA methyltransferase is mammalian DNA methyltransferase. In aspects, the DNA methyltransferase is human DNA methyltransferase. In aspects, the DNA methyltransferase is mouse DNA methyltransferase. In aspects, the DNA methyltransferase is a bacterial cytosine methyltransferase and/or a bacterial non-cytosine methyltransferase. Depending on the specific DNA methyltransferase, different regions of DNA are methylated. For example, Dnmt3A typically targets CpG dinucleotides for methylation. Through DNA methylation, DNA methyltransferases can modify the activity of a DNA segment (e.g., gene expression) without altering the DNA sequence. In aspects, DNA methylation results in repression of gene transcription and/or modulation of methylation sensitive transcription factors. As described herein, fusion proteins may include one or more (e.g., two) DNA metyltransferases. When a DNA methyltransferase is included as part of a fusion protein, the DNA methyltransferase may be referred to as a “DNA methyltransferase domain.” In aspects, a DNA methyltransferase domain includes one or more DNA methyltransferases. In aspects, a DNA methyltransferase domain includes two DNA methyltransferases. In aspects, the DNA methyltransferase domain further comprises a catalytically inactive regulatory factor of DNA methyltransferase (e.g., Dnmt3L) that is needed for the functioning of Dnmtl, Dnmt3A, and Dnmt3B. In aspects, the DNA methyltransferase domain comprises Dnmtl. In aspects, the DNA methyltransferase domain comprises Dnmt3B. In aspects, the DNA methyltransferase domain comprises Dnmt3A. In aspects, the DNA methyltransferase domain further comprises Dnmt3L. In aspects, the DNA methyltransferase domain has the amino acid sequence of SEQ ID NO: 4.

NHDQEFDPPKVYPPVPAEKRKPIRVLSLFDGIATGLLVLKDLGIQVDRY
IASEVCEDSITVGMVRHQGKIMYVGDVRSVTQKHIQEWGPFDLVIGGSP
CNDLSIVNPARKGLYEGTGRLFFEFYRLLHDARPKEGDDRPFFWLFENV
VAMGVSDKRDISRFLESNPVMIDAKEVSAAHRARYFWGNLPGMNRPLAS
TVNDKLELQECLEHGRIAKFSKVRTITTRSNSIKQGKDQHFPVFMNEKE
DILWCTEMERVFGFPVHYTDVSNMSRLARQRLLGRSWSVPVIRHLFAPL
KEYFACV
(SEQ ID NO: 4; (Dnmt3A; residues 612-912; from
Siddique et al., JMB, 2013; Stepper et al.,
NAR, 2016))

In aspects, the DNA methyltransferase domain has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO: 4. In aspects, the DNA methyltransferase domain has an amino acid sequence that has at least 75% sequence identity to SEQ ID NO: 4. In aspects, the DNA methyltransferase domain has an amino acid sequence that has at least 80% sequence identity to SEQ ID NO: 4. In aspects, the DNA methyltransferase domain has an amino acid sequence that has at least 85% sequence identity to SEQ ID NO: 4. In aspects, the DNA methyltransferase domain has an amino acid sequence that has at least 90% sequence identity to SEQ ID NO: 4. In aspects, the DNA methyltransferase domain has an amino acid sequence that has at least 95% sequence identity to SEQ ID NO: 4. In aspects, the DNA methyltransferase domain is Dnmt3L. In aspects, the DNA methyltransferase domain has the amino acid sequence of SEQ ID NO: 5.

MGPMEIYKTVSAWKRQPVRVLSLFRNIDKVLKSLGFLESGSGSGGGILK
YVEDVTNVVRRDVEKWGPFDLVYGSTQPLGSSCDRCPGWYMFQFHRILQ
Y ALPRQESQRPFFWIFMDNLLLTEDDQETTTRFLQTEAVTLQDVRGRD
YQNAMRVWSNIPGLKSKHAPLIPKEEEYLQAQVRSRSKLDAPKVDLLVK
NCLLPLREYFKYFSQNSLPL
(SEQ ID NO: 5 (Dnmt3L; from Siddique et al., JMB,
2013; Stepper et al., NAR, 2016))

In aspects, the DNA methyltransferase domain has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO: 5. In aspects, the DNA methyltransferase domain has an amino acid sequence that has at least 75% sequence identity to SEQ ID NO: 5. In aspects, the DNA methyltransferase domain has an amino acid sequence that has at least 80% sequence identity to SEQ ID NO: 5. In aspects, the DNA methyltransferase domain has an amino acid sequence that has at least 85% sequence identity to SEQ ID NO: 5. In aspects, the DNA methyltransferase domain has an amino acid sequence that has at least 90% sequence identity to SEQ ID NO: 5. In aspects, the DNA methyltransferase domain has an amino acid sequence that has at least 95% sequence identity to SEQ ID NO: 5. In aspects, the DNA methyltransferase domain includes Dnmt3A and Dnmt3L. In aspects, the DNA methyltransferase domain has the amino acid sequence of SEQ ID NO: 6.

NHDQEFDPPKVYPPVPAEKRKPIRVLSLFDGIATGLLVLKDLGIQVDRY
IASEVCEDSITVGMVRHQGKIMYVGDVRSVTQKHIQEWGPFDLVIGGSP
CNDLSIVNPARKGLYEGTGRLFFEFYRLLHDARPKEGDDRPFFWLFENV
VAMGVSDKRDISRFLESNPVMIDAKEVSAAHRARYFWGNLPGMNRPLAS
TVNDKLELQECLEHGRIAKFSKVRIITTRSNSIKQGKDQHFPVFMNEKE
DILWCTEMERVFGFPVHYTDVSNMSRLARQRLLGRSWSVPVIRHLFAPL
KEYFACVSSGNSNANSRGPSFSSGLVPLSLRGSHMGPMEIYKTVSAWKR
QPVRVLSLERNIDKVLKSLGFLESGSGSGGGTLKYVEDVINVVRRDVEK
WGPFDLVYGSTQPLGSSCDRCPGWYMFQFHRILQY ALPRQESQRPFFW
IFMDNLLLTEDDQETTTRFLQTEAVTLQDVRGRDYQNAMRVWSNIPGLK
SKHAPLTPKEEEYLQAQVRSRSKLDAPKVDLLVKNCLLPLREYFKYFSQ
NSLPL
(SEQ ID NO: 6 (Dnmt3A-Dnmt3L domain))

In aspects, the DNA methyltransferase domain has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO: 6. In aspects, the DNA methyltransferase domain has an amino acid sequence that has at least 75% sequence identity to SEQ ID NO: 6. In aspects, the DNA methyltransferase domain has an amino acid sequence that has at least 80% sequence identity to SEQ ID NO:6. In aspects, the DNA methyltransferase domain has an amino acid sequence that has at least 85% sequence identity to SEQ ID NO: 6. In aspects, the DNA methyltransferase domain has an amino acid sequence that has at least 90% sequence identity to SEQ ID NO:6. In aspects, the DNA methyltransferase domain has an amino acid sequence that has at least 95% sequence identity to SEQ ID NO: 6. In aspects, the DNA methyltransferase domain further comprises the Dnmt3L regulatory factor, as described, for example, in Siddique et al. (Targeted methylation and gene silencing of VEGF-A in human cells by using a designed Dnmt3a-Dnmt3L single-chain fusion protein with increased DNA methylation activity, J. Mol. Biol. 425, 2013 and Stepper et al, Efficient targeted DNA methylation with chimeric dCas9-Dnmt3a-Dnmt3L methyltransferase, Nucleic Acids Res. 45, 2017).

A “Dnmt3A”, “Dnmt3a,” “DNA (cytosine-5)-methyltransferase 3A” or “DNA methyltransferase 3a” protein as referred to herein includes any of the recombinant or naturally occurring forms of the Dnmt3A enzyme or variants or homologs thereof that maintain Dnmt3A enzyme activity (e.g., within at least 50%, 55%, 60%, 65%, 70%, 75%, 80%, 85%, 90%, 91%, 92%, 93%, 94%, 95%, 96%, 97%, 98%, 99% or 100% activity compared to Dnmt3A). In aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring Dnmt3A protein. In aspects, the Dnmt3A protein is substantially identical to the protein identified by the UniProt reference number Q9Y6K1 or a variant or homolog having substantial identity thereto. In aspects, the Dnmt3A polypeptide is encoded by a nucleic acid sequence identified by the NCBI reference sequence Accession number NM_022552, homologs or functional fragments thereof. In aspects, Dnmt3A includes the sequence set forth by SEQ ID NO: 4. In aspects, Dnmt3A is the sequence set forth by SEQ ID NO: 4. In aspects, Dnmt3A has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO: 4. In aspects, the DNA methyltransferase domain has an amino acid sequence that has at least 75% sequence identity to SEQ ID NO:4. In aspects, the DNA methyltransferase domain has an amino acid sequence that has at least 80% sequence identity to SEQ ID NO: 4. In aspects, the DNA methyltransferase domain has an amino acid sequence that has at least 85% sequence identity to SEQ ID NO: 4. In aspects, Dnmt3A has an amino acid sequence that has at least 90% sequence identity to SEQ ID NO:4. In aspects, Dnmt3A has an amino acid sequence that has at least 95% sequence identity to SEQ ID NO: 4. In aspects, Dnmt3A is s Dnmt3A transcript 1 (Dnmt3A1). In aspects, Dnmt3A is s Dnmt3A transcript 2 (Dnmt3A2). In aspects, Dnmt3A is s Dnmt3A transcript 3 (Dnmt3A3). In aspects, is s Dnmt3A transcript 4 (Dnmt3A4).

A “Dnmt3L”, “DNA (cytosine-5)-methyltransferase 3L” or “DNA methyltransferase 3L” protein as referred to herein includes any of the recombinant or naturally occurring forms of the Dnmt3L regulatory factor or variants or homologs thereof that maintain Dnmt3L regulatory activity (e.g., within at least 50%, 55%, 60%, 65%, 70%, 75%, 80%, 85%, 90%, 91%, 92%, 93%, 94%, 95%, 96%, 97%, 98%, 99% or 100% activity compared to Dnmt3L). In aspects, the variants or homologs have at least 50%, 55%, 60%, 65%, 70%, 75%, 80%, 85%, 90%, 91%, 92%, 93%, 94%, 95%, 96%, 97%, 98%, 99% or 100% amino acid sequence identity across the whole sequence or a portion of the sequence compared to a naturally occurring Dnmt3L protein. In aspects, the Dnmt3L protein is substantially identical to the protein identified by the UniProt reference number Q9CWR8 or a variant or homolog having substantial identity thereto. In aspects, the Dnmt3L protein is identical to the protein identified by the UniProt reference number Q9CWR8. In aspects, the Dnmt3L protein has at least 75% sequence identity to the amino acid sequence of the protein identified by the UniProt reference number Q9CWR8. In aspects, the Dnmt3L protein has at least 80% sequence identity to the amino acid sequence of the protein identified by the UniProt reference number Q9CWR8. In aspects, the Dnmt3L protein has at least 85% sequence identity to the amino acid sequence of the protein identified by the UniProt reference number Q9CWR8. In aspects, the Dnmt3L protein has at least 95% sequence identity to the amino acid sequence of the protein identified by the UniProt reference number Q9CWR8.

In aspects, the Dnmt3L protein is substantially identical to the protein identified by the UniProt reference number Q9UJW or a variant or homolog having substantial identity thereto. In aspects, the Dnmt3L protein is identical to the protein identified by the UniProt reference number Q9UJW. In aspects, the Dnmt3L protein has at least 50% sequence identity to the amino acid sequence of the protein identified by the UniProt reference number Q9UJW. In aspects, the Dnmt3L protein has at least 55% sequence identity to the protein identified by the UniProt reference number Q9UJW. In aspects, the Dnmt3L protein has at least 60% sequence identity to the amino acid sequence of the protein identified by the UniProt reference number Q9UJW. In aspects, the Dnmt3L protein has at least 65% sequence identity to the amino acid sequence of the protein identified by the UniProt reference number Q9UJW. In aspects, the Dnmt3L protein has at least 70% sequence identity to the amino acid sequence of the protein identified by the UniProt reference number Q9UJW. In aspects, the Dnmt3L protein has at least 75% sequence identity to the amino acid sequence of the protein identified by the UniProt reference number Q9UJW. In aspects, the Dnmt3L protein has at least 80% sequence identity to the amino acid sequence of the protein identified by the UniProt reference number Q9UJW. In aspects, the Dnmt3L protein has at least 85% sequence identity to the amino acid sequence of the protein identified by the UniProt reference number Q9UJW. In aspects, the Dnmt3L protein has at least 90% sequence identity to the amino acid sequence of the protein identified by the UniProt reference number Q9UJW. In aspects, the Dnmt3L protein has at least 95% sequence identity to the amino acid sequence of the protein identified by the UniProt reference number Q9UJW. In aspects, the Dnmt3L polypeptide is encoded by a nucleic acid sequence identified by the NCBI reference sequence Accession number NM_001081695, or homo logs or functional fragments thereof. In aspects, Dnmt3L includes the sequence set forth by SEQ ID NO: 5. In aspects, Dnmt3L is the sequence set forth by SEQ ID NO: 5. In aspects, Dnmt3L has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO: 5. In aspects, Dnmt3L has an amino acid sequence that has at least 50% sequence identity to SEQ ID NO: 5. In aspects, Dnmt3L has an amino acid sequence that has at least 55% sequence identity to SEQ ID NO: 5. In aspects, Dnmt3L has an amino acid sequence that has at least 60% sequence identity to SEQ ID NO: 5. In aspects, Dnmt3L has an amino acid sequence that has at least 65% sequence identity to SEQ ID NO: 5. In aspects, Dnmt3L has an amino acid sequence that has at least 97% sequence identity to SEQ ID NO: 5. In aspects, Dnmt3L has an amino acid sequence that has at least 75% sequence identity to SEQ ID NO: 5. In aspects, Dnmt3L has an amino acid sequence that has at least 80% sequence identity to SEQ ID NO:5. In aspects, Dnmt3L has an amino acid sequence that has at least 85% sequence identity to SEQ ID NO: 5. In aspects, Dnmt3L has an amino acid sequence that has at least 90% sequence identity to SEQ ID NO:5. In aspects, Dnmt3L has an amino acid sequence that has at least 95% sequence identity to SEQ ID NO: 5.

A “nuclear localization sequence” or “nuclear localization signal” or “NLS” is a peptide that directs proteins to the nucleus. In aspects, the NLS includes five basic, positively charged amino acids. The NLS may be located anywhere on the peptide chain. In aspects, the NLS is an NLS derived from SV 40. In aspects, the NLS includes the sequence set forth by SEQ ID NO: 7: PKKKRKV (SV 40 NLS). In aspects, the NLS is the sequence set forth by SEQ ID NO: 7. In aspects, NLS has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO: 7. In aspects, NLS has an amino acid sequence that has at least 75% sequence identity to SEQ ID NO: 7. In aspects, NLS has an amino acid sequence that has at least 80% sequence identity to SEQ ID NO: 7. In aspects, NLS has an amino acid sequence that has at least 85% sequence identity to SEQ ID NO: 7. In aspects, NLS has an amino acid sequence that has at least 90% sequence identity to SEQ ID NO: 7. In aspects, NLS has an amino acid sequence that has at least 95% sequence identity to SEQ ID NO: 7. In aspects, NLS has an amino acid sequence of SEQ ID NO: 7.

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA Examples of nucleic acids, e.g. polynucleotides, contemplated herein include, but are not limited to, any type of RNA, e.g., mRNA, siRNA, miRNA, sgRNA, and guide RNA and any type of DNA, genomic DNA, plasmid DNA, and minicircle DNA, and any fragments thereof. In aspects, the nucleic acid is messenger RNA In aspects, the messenger RNA is messenger ribonucleoprotein (RNP). The term “duplex” in the context of polynucleotides refers, in the usual and customary sense, to double strandedness. Nucleic acids can be linear or branched. For example, nucleic acids can be a linear chain of nucleotides, or the nucleic acids can be branched, e.g., such that the nucleic acids comprise one or more arms or branches of nucleotides. Optionally, the branched nucleic acids are repetitively branched to form higher ordered structures such as dendrimers and the like.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, sgRNA, guide RNA, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g., phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In aspects, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

The term “complementary” or “complementarity” refers to the ability of a nucleic acid to form hydrogen bond(s) with another nucleic acid sequence by either traditional Watson-Crick or other non-traditional types. For example, the sequence A-G-T is complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 10, 15, 20, 25, 30, 35, 40, 45, 50, or more nucleotides, or refers to two nucleic acids that hybridize under stringent conditions (i.e., stringent hybridization conditions).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in IX SSC at 45° C. A positive hybridization is at least twice background. One of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, ed. Ausubel, et al., supra.

The term “gene” means the segment of DNA involved in producing a protein; it can include regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules may be detected by standard PCR or Northern blot methods well known in the art. See, Sambrook et al., 1989 Molecular Cloning: A Laboratory Manual, 18.1-18. 88.

The term “transcriptional regulatory sequence” as provided herein refers to a segment of DNA that is capable of increasing or decreasing transcription (e.g., expression) of a specific gene within an organism. Non-limiting examples of transcriptional regulatory sequences include promoters, enhancers, and silencers.

The terms “transcription start site” and transcription initiation site” may be used interchangeably to refer herein to the 5′ end of a gene sequence (e.g., DNA sequence) where RNA polymerase (e.g., DNA-directed RNA polymerase) begins synthesizing the RNA transcript. The transcription start site may be the first nucleotide of a transcribed DNA sequence where RNA polymerase begins synthesizing the RNA transcript. A skilled artisan can determine a transcription start site via routine experimentation and analysis, for example, by performing a run-off transcription assay or by definitions according to FANTOM5 database.

The term “promoter” as used herein refers to a region of DNA that initiates transcription of a particular gene. Promoters are typically located near the transcription start site of a gene, upstream of the gene and on the same strand (i.e., 5′ on the sense strand) on the DNA Promoters may be about 100 to about 1000 base pairs in length.

The term “enhancer” as used herein refers to a region of DNA that may be bound by proteins (e.g., transcription factors) to increase the likelihood that transcription of a gene will occur. Enhancers may be about 50 to about 1500 base pairs in length. Enhancers may be located downstream or upstream of the transcription initiation site that it regulates and may be several hundreds of base pairs, several thousands of base pairs, or several millions of base pairs away from the transcription initiation site.

The term “silencer” as used herein refers to a DNA sequence capable of binding transcription regulation factors known as repressors, thereby negatively effecting transcription of a gene. Silencer DNA sequences may be found at many different positions throughout the DNA, including, but not limited to, upstream of a target gene for which it acts to repress transcription of the gene (e.g., silence gene expression).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides may also be referred to by their commonly accepted single-letter codes.

The terms “polypeptide,” “peptide” and “protein” are used interchangeably herein to refer to a polymer of amino acid residues, wherein the polymer may, in aspects, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

A “cell” as used herein, refers to a cell carrying out metabolic or other function sufficient to preserve or replicate its genomic DNA A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., Spodoptera) and human cells. Cells may be useful when they are naturally nonadherent or have been treated not to adhere to surfaces, for example by trypsinization.

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cell type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

The terms “transfection”, “transduction”, “transfecting” or “transducing” can be used interchangeably and are defined as a process of introducing a nucleic acid molecule (e.g., mRNA, DNA, RNP) and/or a protein to a cell. Nucleic acids may be introduced to a cell using non-viral or viral-based methods. The nucleic acid molecule can be a sequence encoding complete proteins or functional portions thereof. Typically, a nucleic acid vector, comprising the elements necessary for protein expression (e.g., a promoter, transcription start site, etc.). Nonviral methods of transfection include any appropriate method that does not use viral DNA or viral particles as a delivery system to introduce the nucleic acid molecule into the cell. Exemplary non-viral transfection methods include nanoparticle encapsulation of the nucleic acids that encode the fusion protein (e.g., lipid nanoparticles, gold nanoparticles, and the like), calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral and adeno-associated viral vectors. In aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4: 119-20.

A “peptide linker” as provided herein is a linker including a peptide moiety. In embodiments, the peptide linker is a divalent peptide, such as an amino acid sequence attached at the N-terminus and the C-terminus to the remainder of the compound (e.g., fusion protein provided herein. The peptide linker may be a peptide moiety (a divalent peptide moiety) capable of being cleaved (e.g., a P2A cleavable polypeptide). A peptide linker as provided herein may also be referred to interchangeably as an amino acid linker. In aspects, the peptide linker includes 1 to about 80 amino acid residues. In aspects, the peptide linker includes 1 to about 70 amino acid residues. In aspects, the peptide linker includes 1 to about 60 amino acid residues. In aspects, the peptide linker includes 1 to about 50 amino acid residues. In aspects, the peptide linker includes 1 to about 40 amino acid residues. In aspects, the peptide linker includes 1 to about 30 amino acid residues. In aspects, the peptide linker includes 1 to about 25 amino acid residues. In aspects, the peptide linker includes 1 to about 20 amino acid residues. In aspects, the peptide linker includes about 2 to about 20 amino acid residues. In aspects, the peptide linker includes about 2 to about 19 amino acid residues. In aspects, the peptide linker includes about 2 to about 18 amino acid residues. In aspects, the peptide linker includes about 2 to about 17 amino acid residues. In aspects, the peptide linker includes about 2 to about 16 amino acid residues. In aspects, the peptide linker includes about 2 to about 15 amino acid residues. In aspects, the peptide linker includes about 2 to about 14 amino acid residues. In aspects, the peptide linker includes about 2 to about 13 amino acid residues. In aspects, the peptide linker includes about 2 to about 12 amino acid residues. In aspects, the peptide linker includes about 2 to about 11 amino acid residues. In aspects, the peptide linker includes about 2 to about 10 amino acid residues. In aspects, the peptide linker includes about 2 to about 9 amino acid residues. In aspects, the peptide linker includes about 2 to about 8 amino acid residues. In aspects, the peptide linker includes about 2 to about 7 amino acid residues. In aspects, the peptide linker includes about 2 to about 6 amino acid residues. In aspects, the peptide linker includes about 2 to about 5 amino acid residues. In aspects, the peptide linker includes about 2 to about 4 amino acid residues. In aspects, the peptide linker includes about 2 to about 3 amino acid residues. In aspects, the peptide linker includes about 3 to about 19 amino acid residues. In aspects, the peptide linker includes about 3 to about 18 amino acid residues. In aspects, the peptide linker includes about 3 to about 17 amino acid residues. In aspects, the peptide linker includes about 3 to about 16 amino acid residues. In aspects, the peptide linker includes about 3 to about 15 amino acid residues. In aspects, the peptide linker includes about 3 to about 14 amino acid residues. In aspects, the peptide linker includes about 3 to about 13 amino acid residues. In aspects, the peptide linker includes about 3 to about 12 amino acid residues. In aspects, the peptide linker includes about 3 to about 11 amino acid residues. In aspects, the peptide linker includes about 3 to about 10 amino acid residues. In aspects, the peptide linker includes about 3 to about 9 amino acid residues. In aspects, the peptide linker includes about 3 to about 8 amino acid residues. In aspects, the peptide linker includes about 3 to about 7 amino acid residues. In aspects, the peptide linker includes about 3 to about 6 amino acid residues. In aspects, the peptide linker includes about 3 to about 5 amino acid residues. In aspects, the peptide linker includes about 3 to about 4 amino acid residues. In aspects, the peptide linker about 16 amino acid residues. In aspects, the peptide linker includes about 17 amino acid residues. In aspects, the peptide linker includes about 18 amino acid residues. In aspects, the peptide linker includes about 19 amino acid residues. In aspects, the peptide linker includes about 20 amino acid residues. In aspects, the peptide linker includes about 21 amino acid residues. In aspects, the peptide linker includes about 22 amino acid residues. In aspects, the peptide linker includes about 23 amino acid residues. In aspects, the peptide linker includes about 24 amino acid residues. In aspects, the peptide linker includes about 25 amino acid residues. includes about 10 to about 20 amino acid residues. In aspects, the peptide linker includes about 15 to about 20 amino acid residues. In aspects, the peptide linker includes about 2 amino acid residues. In aspects, the peptide linker includes about 3 amino acid residues. In aspects, the peptide linker includes about 4 amino acid residues. In aspects, the peptide linker includes about 5 amino acid residues. In aspects, the peptide linker includes about 6 amino acid residues. In aspects, the peptide linker includes about 7 amino acid residues. In aspects, the peptide linker includes about 8 amino acid residues. In aspects, the peptide linker includes about 9 amino acid residues. In aspects, the peptide linker includes about 10 amino acid residues. In aspects, the peptide linker includes about 11 amino acid residues. In aspects, the peptide linker includes about 12 amino acid residues. In aspects, the peptide linker includes about 13 amino acid residues. In aspects, the peptide linker includes about 14 amino acid residues. In aspects, the peptide linker includes about 15 amino acid residues. In aspects, the peptide linker includes about 16 amino acid residues. In aspects, the peptide linker includes about 17 amino acid residues. In aspects, the peptide linker includes about 18 amino acid residues. In aspects, the peptide linker includes about 19 amino acid residues. In aspects, the peptide linker includes about 20 amino acid residues. In aspects, the peptide linker includes about 21 amino acid residues. In aspects, the peptide linker includes about 22 amino acid residues. In aspects, the peptide linker includes about 23 amino acid residues. In aspects, the peptide linker includes about 24 amino acid residues. In aspects, the peptide linker includes about 25 amino acid residues.

In aspects, the peptide linker includes the sequence set forth by SEQ ID NO: 8; GGSGGGS. In aspects, the peptide linker is the sequence set forth by SEQ ID NO: 8. In aspects, the peptide linker includes the sequence set forth by SEQ ID NO: 9; SGS. In aspects, the peptide linker is the sequence set forth by SEQ ID NO: 9. In aspects, the peptide linker includes the sequence set forth by SEQ ID NO: 10; EASGSGRASPGIPGSTR. In aspects, the peptide linker is the sequence set forth by SEQ ID NO: 10. In aspects, the peptide linker includes the sequence set forth by SEQ ID NO: 11; SRAD. In aspects, the peptide linker is the sequence set forth by SEQ ID NO: 11. In aspects, the peptide linker includes the sequence set forth by SEQ ID NO: 12; GSG. In aspects, the peptide linker is the sequence set forth by SEQ ID NO: 12. In aspects, the peptide linker includes the sequence set forth by SEQ ID NO: 13; SPG. In aspects, the peptide linker is the sequence set forth by SEQ ID NO: 13. In aspects, the peptide linker includes the sequence set forth by SEQ ID NO: 14; SGNSNANSRGPSFSSGLVPLSLRGSH. In aspects, the peptide linker is the sequence set forth by SEQ ID NO:14. In aspects, the peptide linker includes the sequence set forth by SEQ ID NO: 15; YPYDVPDYA. In aspects, the peptide linker is the sequence set forth by SEQ ID NO: 15. In aspects, the peptide linker includes the sequence set forth by SEQ ID NO: 16; ATNFSLLKQAGDVEENPGP (P2A peptide cleave sequence). In aspects, the peptide linker is the sequence set forth by SEQ ID NO: 16. In aspects, the peptide linker is an XTEN polypeptide. In aspects, the peptide linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO: 7, 8, 9, 10, 11, 12, 13, 14, 15 or 16. In aspects, the peptide linker has an amino acid sequence that has at least 90% sequence identity to SEQ ID NO: 7, 8, 9, 10, 11, 12, 13, 14, 15 or 16.

In aspects, the peptide linker has an amino acid sequence that has at least 90% sequence identity to SEQ ID NO: 17; GGSGGGS. In aspects, the peptide linker has an amino acid sequence that has at least 90% sequence identity to SEQ ID NO: 18, SGS. In aspects, the peptide linker has an amino acid sequence that has at least 90% sequence identity to SEQ ID NO: 19; EASGSGRASPGIPGSTR. In aspects, the peptide linker has an amino acid sequence that has at least 90% sequence identity to SEQ ID NO: 20; SRAD. In aspects, the peptide linker has an amino acid sequence that has at least 90% sequence identity to SEQ ID NO: 21; GSG. In aspects, the peptide linker has an amino acid sequence that has at least 90% sequence identity to SEQ ID NO: 22; SPG. In aspects, the peptide linker has an amino acid sequence that has at least 90% sequence identity to SEQ ID NO: 23; YPYDVPDYA. In aspects, the peptide linker has an amino acid sequence that has at least 90% sequence identity to SEQ ID NO: 24; SSGNSNANSRGPSFSSGLVPLSLRGSH. In aspects, the peptide linker has an amino acid sequence that has at least 90% sequence identity to SEQ ID NO: 25; ATNFSLLKQAGDVEENPGP. In aspects, the peptide linker has an amino acid sequence that has at least 95% sequence identity to SEQ ID NO: 17, 18, 19, 20, 21, 22, 23, 24 or 25. In aspects, the peptide linker has an amino acid sequence that has at least 95% sequence identity to SEQ ID NO: 17. In aspects, the peptide linker has an amino acid sequence that has at least 95% sequence identity to SEQ ID NO: 18. In aspects, the peptide linker has an amino acid sequence that has at least 95% sequence identity to SEQ ID NO: 19. In aspects, the peptide linker has an amino acid sequence that has at least 95% sequence identity to SEQ ID NO: 20. In aspects, the peptide linker has an amino acid sequence that has at least 95% sequence identity to SEQ ID NO: 21. In aspects, the peptide linker has an amino acid sequence that has at least 95% sequence identity to SEQ ID NO: 22. In aspects, the peptide linker has an amino acid sequence that has at least 95% sequence identity to SEQ ID NO: 23. In aspects, the peptide linker has an amino acid sequence that has at least 95% sequence identity to SEQ ID NO: 24. In aspects, the peptide linker has an amino acid sequence that has at least 95% sequence identity to SEQ ID NO: 25.

The terms “XTEN,” “XTEN linker,” or “XTEN polypeptide” as used herein refer to a recombinant polypeptide (e.g. unstructured recombinant peptide) lacking hydrophobic amino acid residues. The development and use of XTEN can be found in, for example, Schellenberger et al., Nature Biotechnology 27, 1186-1190 (2009). In aspects, the XTEN linker includes the sequence set forth by SEQ ID NO: 26; SGSETPGTSESATPES. In aspects, the XTEN linker is the sequence set forth by SEQ ID NO: 26. In aspects, the XTEN linker includes the sequence set forth by SEQ IDNO: 27; GGPSSGAPPPSGGSPAGSPTSTEEGTSESATPESGPGTSTEPSEGSAPGSPAGSPTSTEEGTS TEPSEGSAPGTSTEPSE. In aspects, the XTEN linker is the sequence set forth by SEQ ID NO:27.

In aspects, the XTEN linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO: 26. In aspects, the XTEN linker has an amino acid sequence that has at least 90% sequence identity to SEQ ID NO: 26. In aspects, the XTEN linker has an amino acid sequence that has at least 95% sequence identity to SEQ ID NO:26. In aspects, the XTEN linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO: 27.

In aspects, the XTEN linker has an amino acid sequence that has at least 90% sequence identity to SEQ ID NO: 27. In aspects, the XTEN linker has an amino acid sequence that has at least 95% sequence identity to SEQ ID NO: 27.

“Epitope tag” refers to a biological moiety, such as a peptide, that is genetically engineered into a recombinant protein and that functions as a universal epitope that is easily detected by commercially available assays or antibodies and that generally does not compromise the native structure or function of the protein.

A “detectable agent” or “detectable moiety” is a composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹²⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monocrystalline iron oxide nanoparticles, monocrystalline iron oxide, nanoparticle contrast agents, liposomes or other delivery vehicles containing Gadolinium chelate (“Gd-chelate”) molecules, Gadolinium, radioisotopes, radionuclides (e.g., carbon-I 1, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gases, perfluorcarbon, nitrogen, octafluoropropane, perflexane lipid microsphere, perflutren), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

A detectable moiety is a monovalent detectable agent or a detectable agent capable of forming a bond with another composition. In aspects, the detectable agent is an epitope tag. In aspects, the epitope tag is an HA tag. In aspects, the HA tag includes the sequence set forth by SEQ ID NO: 15 (YPYDVPDYA). In aspects, the HA tag is the sequence set forth by SEQ ID NO: 15. In aspects, the HA tag has an amino acid sequence that has at least 80% sequence identity to SEQ ID NO: 15. In aspects, the HA tag has an amino acid sequence that has at least 85% sequence identity to SEQ ID NO: 15. In aspects, the HA tag has an amino acid sequence that has at least 90% sequence identity to SEQ ID NO:15. In aspects, the HA tag has an amino acid sequence that has at least 95% sequence identity to SEQ ID NO: 15.

In aspects, the detectable agent is a fluorescent protein. In aspects, the fluorescent protein is blue fluorescent protein (BFP). In aspects, the BFP includes the sequence set forth by SEQ ID NO: 28; SELIKENMHMKLYMEGTVDNHHFKCTSEGEGKPYEGTQTMRIKVVEGGPLPF AFDILATSFLYGSKTFINHTQGIPDFFKQSFPEGFTWERVTTYEDGGVLTATQDTSLQDGCLIYNVKIRGVNFTSNG PVMQKKTLGWEAFTETLYPADGGLEGRNDMALKLVGGSHLIANIKTTYRSKKPAKNLKMPGVYYVDYRLERIKEANN ETYVEQHEVAVARYCDLPSKLGHKLN*. In aspects, the BFP is the sequence set forth by SEQ ID NO: 28. In aspects, the BFP has an amino acid sequence that has at least 80% sequence identity to SEQ ID NO: 28. In aspects, the BFP has an amino acid sequence that has at least 85% sequence identity to SEQ ID NO: 28. In aspects, the BFP has an amino acid sequence that has at least 90% sequence identity to SEQ ID NO: 28. In aspects, the BFP has an amino acid sequence that has at least 95% sequence identity to SEQ ID NO: 28.

Radioactive substances (e.g., radioisotopes) that may be used as imaging and/or labeling agents in accordance with the aspects of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y ⁹⁰Y, ⁸⁹Sr, ⁸⁹Zr, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹²⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac. Paramagnetic ions that may be used as additional imaging agents in accordance with the aspects of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

“Contacting” is used in accordance with its plain ordinary meaning and refers to the process of allowing at least two distinct species to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a fusion protein as provided herein and a nucleic acid sequence (e.g., target DNA sequence).

As defined herein, the term “inhibition”, “inhibit”, “inhibiting,” “repression,” repressing,” “silencing,” “silence” and the like when used in reference to a composition as provided herein (e.g., fusion protein, complex, nucleic acid, vector) refer to negatively affecting (e.g., decreasing) the activity (e.g., transcription) of a nucleic acid sequence (e.g., decreasing transcription of a gene, such as a viral gene) relative to the activity of the nuclei acid sequence (e.g., transcription of a gene) in the absence of the composition (e.g., fusion protein, complex, nucleic acid, vector). In aspects, inhibition refers to reduction of a disease or symptoms of disease (e.g., HIV/AIDS). Thus, inhibition includes, at least in part, partially or totally blocking activation (e.g., transcription), or decreasing, preventing, or delaying activation (e.g., transcription) of the nucleic acid sequence. The inhibited activity (e.g., transcription) may be 90%, 80%, 70%, 60%, 50%, 40%, 30%, 20%, 10%, or less than that in a control. In aspects, the inhibition is 1.5-fold, 2-fold, 3-fold, 4-fold, 5-fold, 10-fold, or more in comparison to a control.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

### Fusion Proteins

Provided herein are, inter alia, fusion proteins that can turn off genes permanently (e.g., irreversibly) and reversibly in mammalian cells using epigenome editing. In embodiments, the fusion protein includes a single polypeptide fusion of proteins (e.g., catalytically inactive Brec (e.g., dBrec1), a KRAB domain, Dnmt3A and Dnmt3L) which can be transiently delivered as mRNA, DNA or RNP and expressed transiently in cells. The fusion protein is directed to a 34-bp sequence present in the long terminal repeats (LTRs) of the majority of the clinically relevant HIV-1 strains and subtypes using dBrec1. Once properly positioned and without intending to be bound by a theory, the fusion protein adds DNA methylation and/or repressive chromatin marks to the target nucleic acid, resulting in gene silencing that is inheritable across subsequent cell divisions. In this way, the fusion protein can perform epigenome editing that bypasses the need to generate DNA double-strand breaks in the host genome, making it a safe and reversible way of manipulating the genome of a living organism.

In embodiments, the fusion protein comprises a catalytically inactive version of Brec1; a KRAB domain, and a DNA methyltransferase domain. In aspects, the fusion protein comprises, from N-terminus to C-terminus, a DNA methyltransferase domain, a catalytically inactive version of Brec1, and KRAB domain. In aspects, the fusion protein comprises, from N-terminus to C-terminus, a KRAB domain, a catalytically inactive version of Brec1, and a DNA methyltransferase domain. In embodiments, the fusion protein further comprises one or more peptide linkers. In aspects, the fusion protein further comprises one or more detectable tags. In aspects, the fusion protein further comprises one or more nuclear localization sequences. In aspects, the fusion protein further comprises one or more peptide linkers, one or more detectable tags, one or more nuclear localization sequences, or a combination of two or more of the foregoing. When the fusion protein comprises one or more peptide linkers, each peptide linker can be the same or different. When the fusion protein comprises one or more detectable tags, each detectable tag can be the same or different. In aspects, the fusion protein comprises from 1 to 10 detectable tags. In aspects, the fusion protein comprises from 1 to 9 detectable tags. In aspects, the fusion protein comprises from 1 to 8 detectable tags. In aspects, the fusion protein comprises from 1 to 7 detectable tags. In aspects, the fusion protein comprises from 1 to 6 detectable tags. In aspects, the fusion protein comprises from 1 to 5 detectable tags. In aspects, the fusion protein comprises from 1 to 4 detectable tags. In aspects, the fusion protein comprises from 1 to 3 detectable tags. In aspects, the fusion protein comprises from 1 to 2 detectable tags. In aspects, the fusion protein comprises 1 detectable tag. In aspects, the fusion protein comprises 2 detectable tags. In aspects, the fusion protein comprises 3 detectable tags. In aspects, the fusion protein comprises 4 detectable tags. In aspects, the fusion protein comprises 5 detectable tags.

In embodiments, the disclosure provides a fusion protein comprising, from N-terminus to C-terminus, a DNA methyltransferase domain, a first XTEN linker, a catalytically inactive version of Brec1, a second XTEN linker, and a Kruppel-associated box domain. In aspects, the first XTEN linker comprises one or more amino acid residues than the second XTEN linker. In embodiments, the fusion protein comprises, from N-terminus to C terminus, a DNA methyltransferase domain, a first XTEN linker comprising from about 5 to about 864 amino acid residues, a catalytically inactive version of Brec1, a second XTEN linker comprising from about 5 to about 864 amino acid residues, and a Kruppel associated box domain. In aspects, the first and second XTEN linkers comprise from about 20 to about 100 amino acid residues. In aspects, the first XTEN linker comprises from about 60 to about 864 amino acid residues, and the second XTEN linker comprises from about 10 to about 40 amino acid residues. In aspects, the first XTEN linker comprises from about 70 to about 864 amino acid residues, and the second XTEN linker comprises from about 10 to about 30 amino acid residues. In aspects, the DNA methyltransferase domain comprises a Dnmt3A. In aspects, the DNA methyltransferase domain (Dnmt3A) further comprises a Dnmt3L regulatory factor (referred to herein as a Dnmt3A-3L domain or a Dnmt3B-3L domain). In aspects, the fusion protein further comprises an epitope tag, a 2A peptide, a fluorescent protein tag, a nuclear localization signal peptide, or a combination of two or more thereof.

In embodiments, the fusion protein comprises, from N-terminus to C-terminus, a DNA methyltransferase domain, a first XTEN linker, a catalytically inactive version of Brec1, an epitope tag, a nuclear localization signal peptide, a second XTEN linker, a Kruppel associated box domain, a 2A cleavable peptide, and a fluorescent protein tag. In aspects, the first XTEN linker comprises one or more amino acid residues than the second XTEN linker. In aspects, the first XTEN linker comprises from greater than 50 to about 864 amino acid residues, and the second XTEN linker comprises from about 5 to 50 amino acid residues. In aspects, the first XTEN linker comprises from about 60 to about 864 amino acid residues, and the second XTEN linker comprises from about 10 to about 40 amino acid residues. In aspects, the first XTEN linker comprises from about 70 to about 864 amino acid residues, and the second XTEN linker comprises from about 10 to about 30 amino acid residues. In aspects, the DNA methyltransferase domain comprises a Dnmt3A domain. In aspects, the Dnmt3A domain is linked to a Dnmt3L regulatory factor (referred to herein as a Dnmt3A-3L domain). In aspects, the DNA methyltransferase domain comprises a Dnmt3B domain. In aspects, the Dnmt3B domain is linked to a Dnmt3L regulatory factor (referred to herein as a Dnmt3B-3L domain).

In embodiments, the fusion protein comprises the structure: A-B-C, or B-A-C or C-A-B, or C-B-A, or B-C-A, or A-C-B; where A comprises a catalytically inactive version of Brec1; B comprises a KRAB domain, C comprises a DNA methyltransferase domain; and wherein the component on the left is the N-terminus and the component on the right is the C-terminus. In aspects, the fusion protein further comprises one or more peptide linkers and one or more detectable tags. In aspects, A-B, B-A, B-C, C-B, A-C, and C-A are each independently linked together via a covalent bond, a peptide linker, a detectable tag, a nuclear localization sequence, or a combination of two or more thereof. The peptide linker can be any known in the art (e.g., P2A cleavable peptide, XTEN linker, and the like). In aspects, the fusion protein comprises other components, such as detectable tags (e.g., HA tag, blue fluorescent protein, and the like).

In embodiments, the fusion protein comprises the structure: A-L₁-B-L₂-C or C-L₂-B-L₁-A or C-L₂-A-L₁-B, where A comprises a catalytically inactive version of Brec1; B comprises a KRAB domain, C comprises a DNA methyltransferase domain, L₁ is absent, a covalent bond, or a peptide linker, and L₂ is absent, a covalent bond, or a peptide linker; and where the component at the left is at the N-terminus and the component on the right is at the C-terminus. In aspects, A is covalently linked to B via a peptide linker. In aspects, A is covalently linked to B via a covalent bond. In aspects, B is covalently linked to C via a peptide linker. In aspects, B is covalently linked to C via a covalent bond. The peptide linker can be any known in the art (e.g., P2A cleavable peptide, XTEN linker, and the like). In aspects, the fusion protein comprises other components, such as detectable tags, nuclear localization sequences, and the like. In aspects, L₁ is a covalent bond, a peptide linker, a detectable tag, a nuclear localization sequence, or a combination thereof. In aspects, L₂ is a covalent bond, a peptide linker, a detectable tag, a nuclear localization sequence, or a combination thereof.

In embodiments, the fusion protein comprises the structure: B-L₁-A-L₂-C or C-L₁-A-L₂-B where A comprise a catalytically inactive version of Brec1; B comprises a KRAB domain, C comprises a DNA methyltransferase domain, L₁ is a covalent bond or a peptide linker, and L₂ is a covalent bond or a peptide linker. In embodiments the fusion protein comprises the structure: B-Li-A-L₂-C. In embodiments the fusion protein comprises the structure: C-L₁-A-L₂-B. In aspects, L₁ is a peptide linker. In aspects, L₁ is a covalent bond. In aspects, L₂ is a peptide linker. In aspects, L₂ is a covalent bond. The peptide linker can be any known in the art or described herein (e.g., P2A cleavable peptide, XTEN linker, and the like). In aspects, the fusion protein comprises other components, such as detectable tags. In aspects, L₁ is a covalent bond, a peptide linker, a detectable tag, or a combination thereof. In aspects, L₂ is a covalent bond, a peptide linker, a detectable tag, or a combination thereof. In aspects, the fusion protein further comprises a nuclear localization sequence.

In embodiments, the fusion protein comprises the structure: B-L₃-A-L₄-C-L₅-D or C-L₃-A-L₄-B-L₅-D, where A comprises a catalytically inactive version of Brec1; B comprises a KRAB domain, C comprises a DNA methyltransferase domain, D is absent or D comprises one or more detectable tags, L₃ comprises a covalent bond, a peptide linker, a detectable tag, or a combination of two or more thereof, L₄ comprises a covalent bond, a peptide linker, a detectable tag, or a combination of two or more thereof, L₅ is absent or L₅ comprises a covalent bond or a peptide linker. In embodiments, the fusion protein comprises the structure: B-L₃-A-L₄-C-L₅-D. In embodiments, the fusion protein comprises the structure: C-L₃-A-L₄-B-L₅-D. In aspects, L₃ is a peptide linker. In aspects, L₃ is a covalent bond. In aspects, L₃ comprises a peptide linker and a detectable tag. In aspects, L₃ comprises a detectable tag. In aspects, L₄ is a peptide linker. In aspects, L₄ comprises a peptide linker and a detectable tag. In aspects, L₄ is a covalent bond. In aspects, L₄ comprises a detectable tag. In aspects, L₅ is a peptide linker. In aspects, L₅ is a covalent bond. In aspects, D comprises one or a plurality of detectable tags. In aspects, D comprises one detectable tag. In aspects, D comprises two detectable tags. In aspects, D comprises three detectable tags. In aspects, D comprises a plurality of detectable tags. D can be any detectable tag known in the art and/or described herein (e.g., HA tag, blue fluorescent protein, and the like). In aspects L₅ and D are absent. When L₃, L₄, L₅, and D comprise two or more detectable tags, each detectable tag is the same or different. The peptide linker can be any known in the art and/or described herein (e.g., P2A cleavable peptide, XTEN linker, and the like). In aspects, the fusion protein further comprises a nuclear localization sequence.

In embodiments, the fusion protein comprises the structure: C-L₃-A-L₄-B-L₅-D, where A comprises a catalytically inactive version of Brec1; B comprises a KRAB domain, C comprises a DNA methyltransferase domain, D is absent or D comprises one or more detectable tags, L₃ comprises a covalent bond, a peptide linker, a detectable tag, or a combination of two or more thereof, L₄ comprises a covalent bond, a peptide linker, a detectable tag, or a combination of two or more thereof, L₅ is absent or L₅ comprises a covalent bond or a peptide linker; and where C is at the N-terminus and Dis at the C-terminus. In aspects, L₃ is a peptide linker. In aspects, L₃ is a covalent bond. In aspects, L₃ comprises a detectable tag. In aspects, L₃ comprises a peptide linker and a detectable tag. In aspects, L₄ a peptide linker. In aspects, L₄ is a covalent bond. In aspects, L₄ comprises a detectable tag. In aspects, L₄ comprises a peptide linker and a detectable tag. In aspects, L₅ a peptide linker. In aspects, L₅ is a covalent bond. In aspects, D comprises one or a plurality of detectable tags. In aspects, D comprises one detectable tag. In aspects, D comprises two detectable tags. In aspects, D comprises three detectable tags. In aspects, D comprises a plurality of detectable tags. D can be any detectable tag known in the art and/or described herein (e.g., HA tag, blue fluorescent protein, and the like). In aspects L₅ and D are absent. When L₃, L₄, L₅, and D comprise two or more detectable tags, each detectable tag is the same or different. The peptide linker can be any known in the art and/or described herein (e.g., P2A cleavable peptide, XTEN linker, and the like). In aspects, the fusion protein further comprises a nuclear localization sequence.

In embodiments, the peptide linker is a XTEN linker. In aspects, the XTEN linker includes about 16 to about 80 amino acid residues. In aspects, the XTEN linker includes about 17 to about 80 amino acid residues. In aspects, the XTEN linker includes about 18 to about 80 amino acid residues. In aspects, the XTEN linker includes about 19 to about 80 amino acid residues. In aspects, the XTEN linker includes about 20 to about 80 amino acid residues. In aspects, the XTEN linker includes about 30 to about 80 amino acid residues. In aspects, the XTEN linker includes about 40 to about 80 amino acid residues. In aspects, the XTEN linker includes about 50 to about 80 amino acid residues. In aspects, the XTEN linker includes about 60 to about 80 amino acid residues. In aspects, the XTEN linker includes about 70 to about 80 amino acid residues. In aspects, the XTEN linker includes about 16 to about 70 amino acid residues. In aspects, the XTEN linker includes about 16 to about 60 amino acid residues. In aspects, the XTEN linker includes about 16 to about 50 amino acid residues. In aspects, the XTEN linker includes about 16 to about 40 amino acid residues. In aspects, the XTEN linker includes about 16 to about 35 amino acid residues. In aspects, the XTEN linker includes about 16 to about 30 amino acid residues. In aspects, the XTEN linker includes about 16 to about 25 amino acid residues. In aspects, the XTEN linker includes about 16 to about 20 amino acid residues. In aspects, the XTEN linker includes about 16 amino acid residues. In aspects, the XTEN linker includes about 17 amino acid residues. In aspects, the XTEN linker includes about 18 amino acid residues. In aspects, the XTEN linker includes about 19 amino acid residues. In aspects, the XTEN linker includes about 20 amino acid residues.

In aspects, the fusion protein comprises at least two XTEN linkers that are the same or different. In aspects, the fusion protein comprises a first XTEN linker having more amino acid residues than a second XTEN linker. In aspects, the fusion protein comprises a first XTEN linker having 10 to 150 amino acid residues than a second XTEN linker. In aspects, the fusion protein comprises a first XTEN linker having 20 to 120 amino acid residues than a second XTEN linker. In aspects, the fusion protein comprises a first XTEN linker having 30 to 110 amino acid residues than a second XTEN linker. In aspects, the fusion protein comprises a first XTEN linker having 40 to 110 amino acid residues than a second XTEN linker. In aspects, the fusion protein comprises a first XTEN linker having 50 to 100 amino acid residues than a second XTEN linker. In aspects, the fusion protein comprises a first XTEN linker having 60 to 100 amino acid residues than a second XTEN linker.

In embodiments, the XTEN linker comprises from about 50 to about 864 amino acid residues. In aspects, the XTEN linker comprises from about 50 to about 200 amino acid residues. In aspects, the XTEN linker comprises from about 55 to about 180 amino acid residues. In aspects, the XTEN linker comprises from about 60 to about 150 amino acid residues. In aspects, the XTEN linker comprises from about 60 to about 120 amino acid residues. In aspects, the XTEN linker comprises from about 60 to about 110 amino acid residues. In aspects, the XTEN linker comprises from about 60 to about 100 amino acid residues. In aspects, the XTEN linker comprises from about 70 to about 90 amino acid residues. In aspects, the XTEN linker comprises from about 75 to about 85 amino acid residues. In aspects, the XTEN linker comprises about 80 amino acid residues. In aspects, when a fusion protein comprises at least two XTEN peptide linkers, then the XTEN linker that comprise from about 50 to about 200 amino acid residues is referred to as a first XTEN peptide linker.

In embodiments, the XTEN linker comprises from about 5 to about 55 amino acid residues. In aspects, the XTEN linker comprises from about 5 to about 50 amino acid residues. In aspects, the XTEN linker comprises from about 5 to about 40 amino acid residues. In aspects, the XTEN linker comprises from about 10 to about 30 amino acid residues. In aspects, the XTEN linker comprises from about 10 to about 25 amino acid residues. In aspects, the XTEN linker comprises from about 10 to about 20 amino acid residues. In aspects, the XTEN linker comprises from about 14 to about 18 amino acid residues. In aspects, the XTEN linker comprises about 16 amino acid residues. In aspects, when a fusion protein comprises at least two XTEN peptide linkers, then the XTEN linker that comprise from about 5 to about 55 amino acid residues is referred to as a second XTEN peptide linker.

The fusion protein may include amino acid sequences useful for targeting the fusion protein to specific regions of a cell (e.g., cytoplasm, nucleus). Thus, in aspects, the fusion protein further includes a nuclear localization signal (NLS, such as the SV40 NLS) peptide. In aspects, the NLS includes the sequence set forth by SEQ ID NO: 7. In aspects, the NLS is the sequence set forth by SEQ ID NO: 7. In aspects, the NLS has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO: 7. In aspects, the NLS has an amino acid sequence that has at least 75% sequence identity to SEQ ID NO: 7. In aspects, the NLS has an amino acid sequence that has at least 80% sequence identity to SEQ ID NO: 7. In aspects, the NLS has an amino acid sequence that has at least 85% sequence identity to SEQ ID NO: 7. In aspects, the NLS has an amino acid sequence that has at least 90% sequence identity to SEQ ID NO: 7. In aspects, the NLS has an amino acid sequence that has at least 95% sequence identity to SEQ ID NO: 7.

In embodiments, the fusion protein includes, from N-terminus to C-terminus, a KRAB domain, a catalytically inactive version of Brec1, and a DNA methyltransferase domain. In embodiments, the DNA methyltransferase domain is a Dnmt3A-3L domain. In embodiments, a catalytically inactive version of Brec1 is dBrec1 and the DNA methyltransferase domain is a Dnmt3A-3L domain. In embodiments, the dBrec1 is covalently linked to the KRAB domain via a peptide linker and wherein the dBrec1 is covalently linked to the Dnmt3A-3L domain via a peptide linker.

### Complexes

In order for the fusion protein to carry out epigenome editing, the fusion protein interacts with (e.g. is non-covalently bound to) a polynucleotide, here HIV LTR.

### Nucleic Acids and Vectors

The fusion protein described herein, including embodiments and aspects thereof, may be provided as a nucleic acid sequence that encodes for the fusion protein. Thus, in an aspect is provided a nucleic acid sequence encoding the fusion protein described herein, including embodiments and aspects thereof. In an aspect is provided a nucleic acid sequence encoding the fusion protein described herein, including embodiments and aspects thereof. In aspects, the nucleic acid sequence encodes for a fusion protein described herein, including fusion proteins having amino acid sequences with certain % sequence identities described herein. In aspects, the nucleic acid is RNA. In aspects, the nucleic acid is messenger RNA. In aspects, fusion protein is delivered as DNA, mRNA, protein or an RNP. In aspects, the nucleic acid sequence encodes for the fusion proteins described herein, including embodiments and aspects thereof.

It is further contemplated that the nucleic acid sequence encoding the fusion protein as described herein, including embodiments and aspects thereof, may be included in a vector. Therefore, in an aspect is provided a vector including a nucleic acid sequence as described herein, including embodiments and aspects thereof. In aspects, the vector comprises a nucleic acid sequence that encodes for a fusion protein described herein, including fusion proteins having amino acid sequences with certain % sequence identities described herein. In aspects, the nucleic acid is messenger RNA. In aspects, the messenger RNA is messenger RNP.

Thus, one or more vectors may include all necessary components for preforming epigenome editing.

### Cells

The compositions described herein may be incorporated into a cell. Inside the cell, the compositions as described herein, including embodiments and aspects thereof, may perform epigenome editing. Accordingly, in an aspect is provided a cell including a fusion protein as described herein, including embodiments and aspects thereof, a nucleic acid as described herein, including embodiments and aspects thereof, a complex as described herein, including embodiments and aspects thereof, or a vector as described herein, including embodiments and aspects thereof. In aspects is provided a cell including a fusion protein as described herein, including embodiments and aspects thereof. In aspects is provided a cell including a nucleic acid as described herein, including embodiments and aspects thereof. In aspects is provided a cell including a complex as described herein, including embodiments and aspects thereof. In aspects is provided a cell including a vector as described herein, including embodiments and aspects thereof. In aspects, the cell is a eukaryotic cell. In aspects, the cell is a mammalian cell.

### Methods

The fusion proteins described herein program a durable memory of gene silencing over time. In embodiments, the disclosure provides methods of silencing a target nucleic acid sequence in a cell, here HIV LTR, the method comprising: (i) delivering a polynucleotide encoding a fusion protein, as described herein, including all embodiments and aspects thereof (e.g., comprising a catalytically inactive version of Brec1), to a cell containing the target nucleic acid; thereby silencing the target nucleic acid sequence. In embodiments, the disclosure provides methods of silencing a target nucleic acid sequence in a cell, the method comprising delivering a polynucleotide encoding a fusion protein, as described herein, including all embodiments and aspects thereof (e.g., comprising a catalytically inactive version of Brec1), to a cell containing the target nucleic acid; thereby silencing the target nucleic acid sequence. In aspects, the method of silencing the target nucleic acid sequence is a method of treating a viral infection in a patient in need thereof. In aspects, the method of silencing the target nucleic acid sequence is a method of treating an infectious disease in a patient in need thereof.

In embodiments, the disclosure provides methods of treating an infectious disease in a subject in need thereof, the method comprising: (i) delivering to the subject an effective amount of a polynucleotide encoding a fusion protein, as described herein, including all embodiments and aspects thereof (e.g., comprising a catalytically inactive version of Brec1); thereby treating the infectious disease in the subject.

In aspects, the sequence within the target nucleic acid sequence is methylated. In aspects, the sequence that is within about 3000, 2900, 2800, 2700, 2600, 2500, 2400, 2300, 2200, 2100, 2000, 1900, 1800, 1700, 1600, 1500, 1400, 1300, 1200, 1100, 1000, 900,800, 700, 600, 500, 400, 300, 200, 100, 50, 20, or 10 base pairs of the target nucleic acid sequence is methylated. Without intending to be bound by any theory, methylating a chromatin can mean that DNA is methylated at the C nucleotide of CG sequences found in CpG islands or non-CpG islands (i.e., adding methyl marks at the C nucleotide of CG DNA sites found in CpG islands).

In embodiments, silencing refers to a complete suppression of transcription. In aspects, silencing refers to a significant decrease in transcription compared to control levels of transcription, such as about 95%, 90%, 85%, 80%, 75%, 70%, 65%, 60%, 55%, 50%, 45%, 40%, 35%, 30%, 25%, 20%, 15% or 10% decrease in transcription as compared to control levels.

In embodiments, the polynucleotide described herein is delivered into the cell by any method known in the art, for example, by transfection, electroporation or transduction. In aspects, the complex is delivered to the cell via RNA or DNA delivery. In aspects, the complex is delivered into the cell via RNA In aspects, the complex is delivered to the cell via DNA. In aspects, the complex is delivered to the cell via transfection, virus, lipid nanoparticle (LNP) or viral-like particles. In aspects, the complex is delivered to the cell via transfection. In aspects, the complex is delivered to the cell via virus. In aspects, the complex is delivered to the cell via lipid nanoparticle. Methods for delivery complexes into a cell are well known in the art.

### EXAMPLES

The invention is now described with reference to the following examples. These examples are provided for the purpose of illustration only and the invention should in no way be construed as being limited to these examples but rather should be construed to encompass any and all variations that become evident as a result of the teaching provided herein.

### Example 1

The technology described herein allows for, inter alia, permanent silencing of genes in mammalian cells without generating double stranded DNA breaks in the host genome. In embodiments, the central component is a single polypeptide chain composed of catalytically inactive Brec (dBrec1) fused to Dnmt3A, Dnmt3L, and a KRAB domain. The tyrosine at residue 324 in Brec1 was mutated to a phenylalanine in dBrec1. This mutation in CRE destroys its recombinase activity but allows it to maintain its DNA binding activity (dBrec1).

The fusion proteins provided herein can add DNA methylation and/or repressive chromatin marks to the site. The result is gene silencing that is inheritable across subsequent cell divisions. In aspects, the fusion protein provided herein can be expressed transiently, bypassing the use of viral delivery methods to induce permanent silencing.

The fusion proteins provided herein provide a robust long-term or permanent silencing of the HIV promoter, long tandem repeat (LTR) by epigenome editing rather than genome editing. An advantage of the fusion protein provided herein is that epigenetic editing is reversible and therefore inherently safer than genome editing. Thus, the fusion proteins provided herein are useful in prophylactic applications. For example, gene silencing can enable acute protection from an infection and then be reversed after the risk of infection or intoxication is absent. The fusion proteins provided herein are useful in genome editing based therapeutics for HIV.

Permanent gene silencing in mammalian cells can be accomplished with a single polypeptide chain composed of dBrec1 fused to DNMT3A, cofactor DNMT3L and the Krüppel associated box (KRAB) domain—herein called BrecOFF.

BrecOFF is an epigenome editor that specifically targets the HIV promoter, long tandem repeat (LTR), to deposit DNA and histone repressive markers and thereby silencing its transcription and reactivation. BrecOFF consist of 3 modules (FIG. 1). The DNMT3A and cofactor DNMT3L are the de novo DNA methylation machinery that marks DNA with repressive methylation marks. The Krüppel associated box (KRAB) domain is a transcriptional repression domain by recruit of TRIM28 repression complex and addition of repressive epigenetic marks on histones. Finally, dBrec1 is a catalytically inactive version of Brec1, a Cre-based directly evolved recombinase that specifically recognizes the R region of the HIV LTR without off-target effects (PMID: 26900663). BrecOFF was constructed by replacing CRISPRoff's Cas9 (Nunez et al. Cell 2021 Apr. 29; 184(9):2503-2519.e17. doi: 10.1016/j.cell.2021.03.025. Epub 2021 Apr. 9) with dBrec1. Alternative version of modules such as fluorescent reporters or other KRAB domains have are also of use in the instant invention.

Compared to other epigenome editors, BrecOFF has an advantage due to its specificity and size. dBrec1 specifically directs the protein to a highly conserved region of HIV (over 90% sequence conservation based on Los Alamos HIV database). BrecOFF is also relatively small 3,987 base pair/148.3 kDa. Both of these characteristics facilitate the delivery of BrecOFF as genetic material or protein into the desired target cell or tissue.

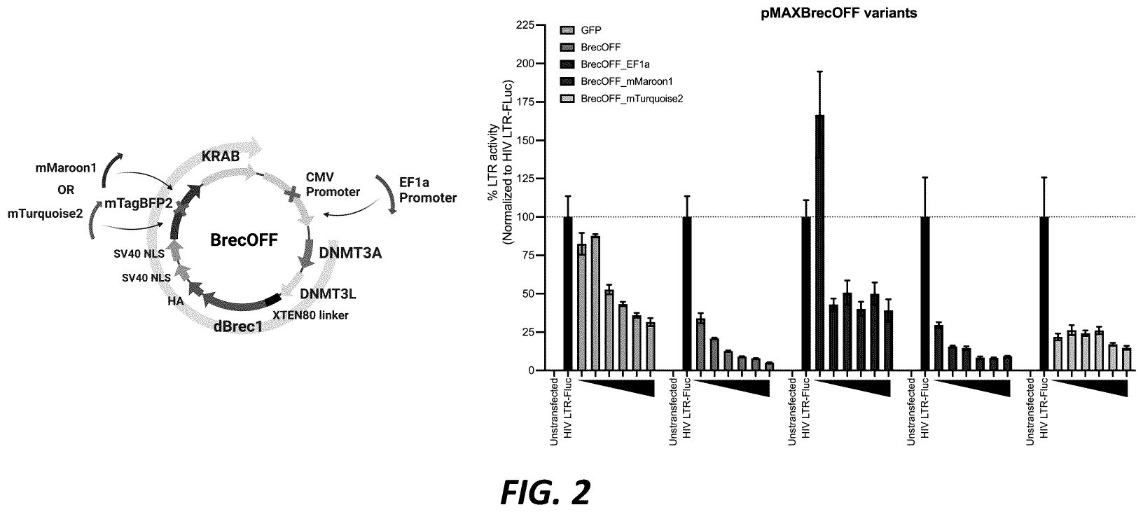

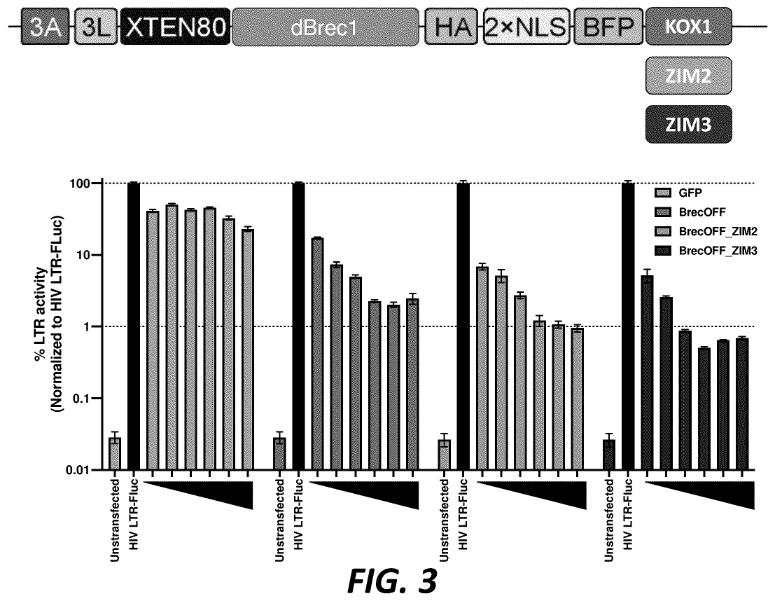

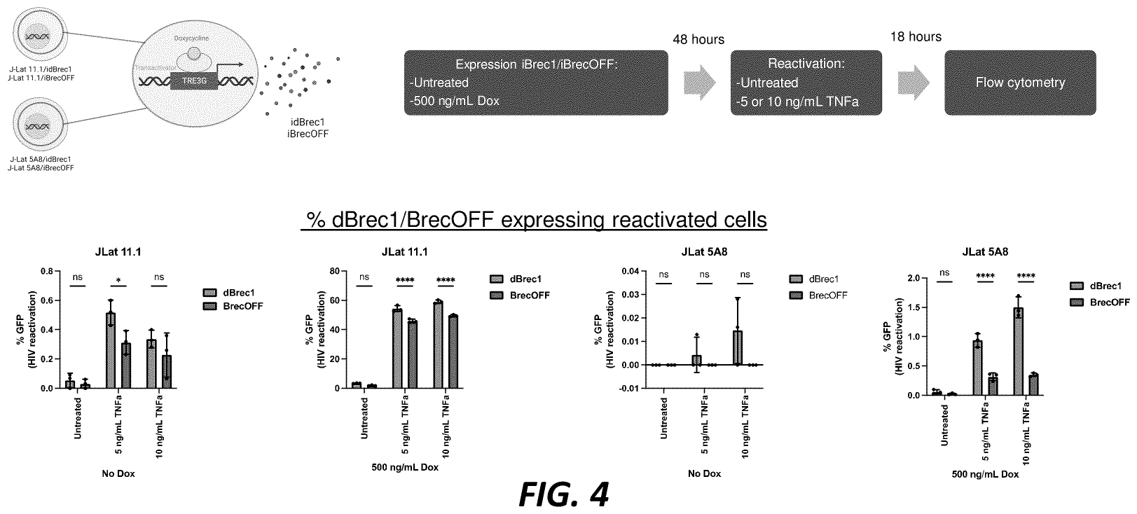

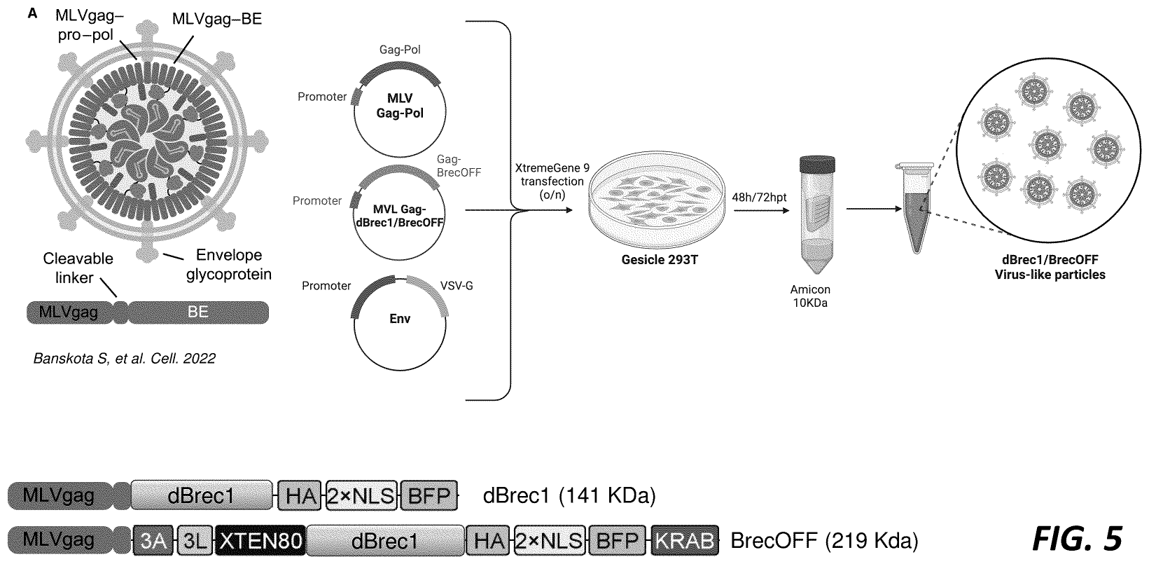

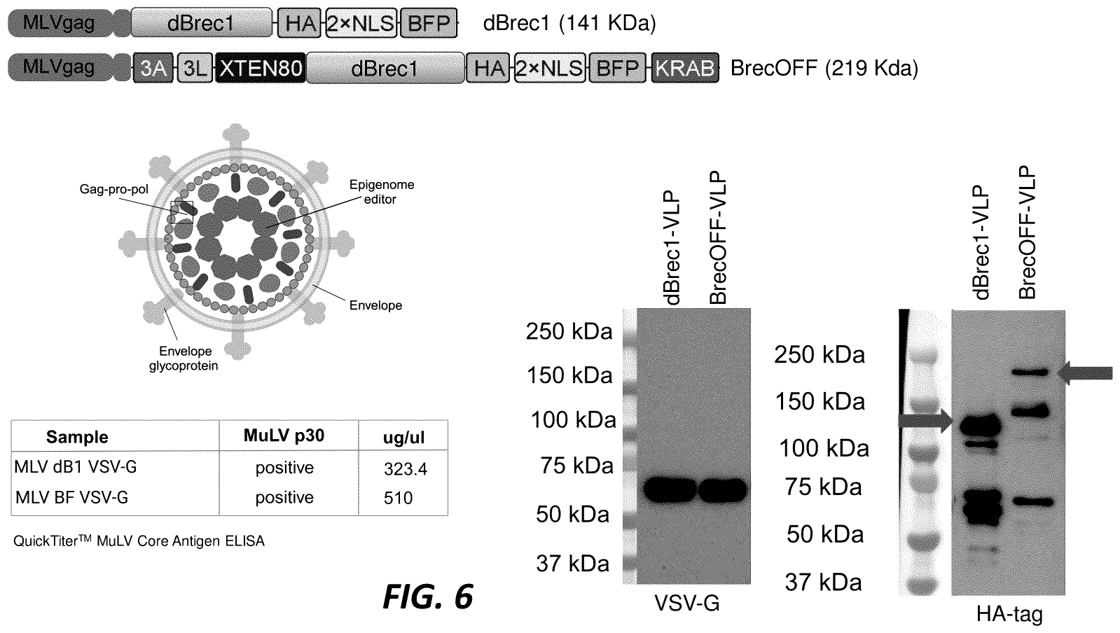

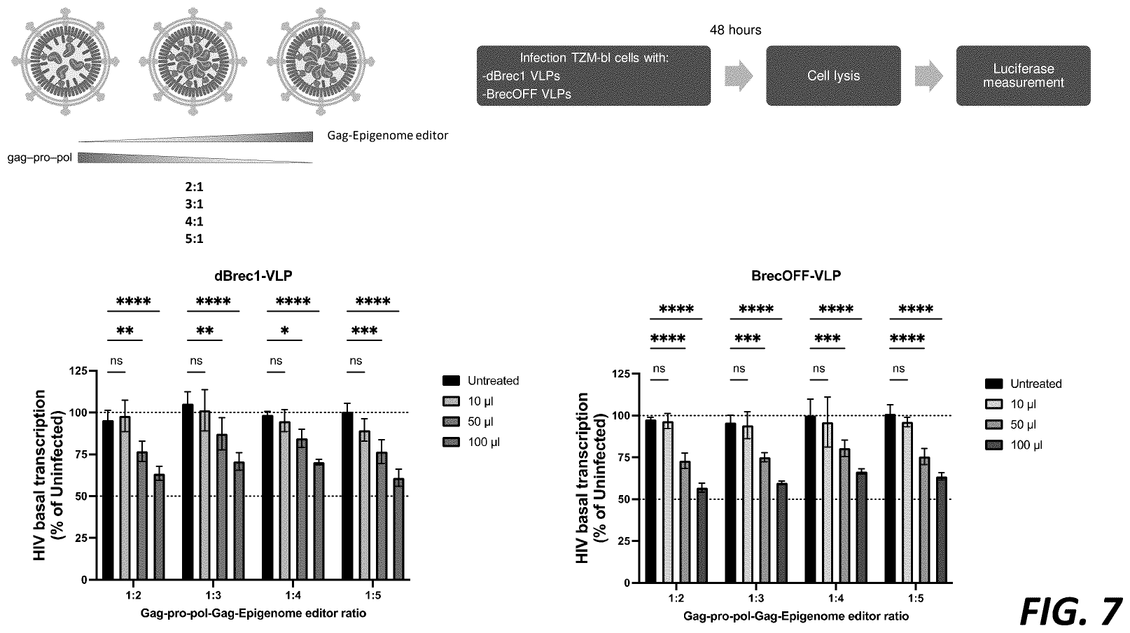

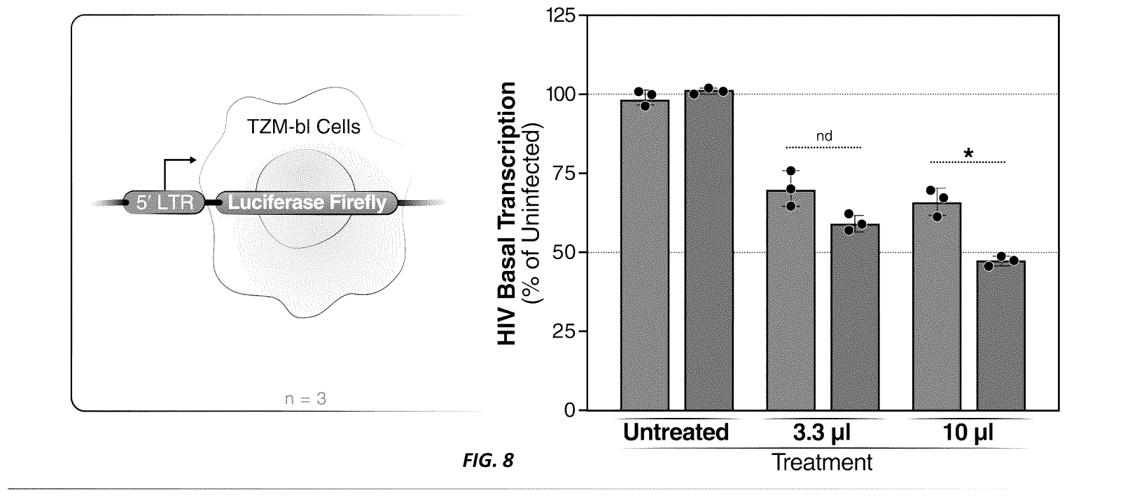

As demonstrated in the drawings, such FIG. 2, BrecOFF represses HIV LTR activity in a dose-dependent manner. Thus, BrecOFF methylates and represses HIV LTR.

### CONCLUSIONS

BrecOFF strongly binds HIV LTR and silences by promoter occupancy, durably represses HIV basal transcription by epigenetic modifications, depends on both DNA and histone methylation for repression and can be packaged in VLPs that repress HIV transcription.

Virus-like particles (VLPs) are molecules that closely resemble viruses but are non-infectious because they contain no viral genetic material. They can be naturally occurring or synthesized through the individual expression of viral structural proteins, which can then self-assemble into the virus-like structure. Combinations of structural capsid proteins from different viruses can be used to create recombinant VLPs. Both in vivo assembly (i.e., assembly inside E. coli bacteria via recombinant co-expression of multiple proteins) and in vitro assembly (i.e., protein self-assembly in a reaction vessel using stoichiometric quantities of previously purified proteins) have been successfully shown to form virus-like particles.

VLPs derived from the Hepatitis B virus (HBV) and composed of the small HBV derived surface antigen (HBsAg) were described in 1968 from patient sera. VLPs have been produced from components of a wide variety of virus families including Parvoviridae (e.g. adeno-associated virus), Retroviridae (e.g. HIV), Flaviviridae (e.g. Hepatitis C virus), Paramyxoviridae (e.g. Nipah) and bacteriophages (e.g. Qβ, AP205). VLPs can be produced in multiple cell culture systems including bacteria, mammalian cell lines, insect cell lines, yeast and plant cells.

BrecOFF can also be administered in a variety other delivery vehicles nanoparticle/microparticle (e.g., liposome, polymeric nanoparticle, lipid based nanoparticle (LNP; e.g., ionizable lipid, a phospholipid, cholesterol and a PEGylated lipid), inorganic nanoparticle or biodegradable polymer (e.g., made from poly(lactic-co-glycolic acid) (PLGA), polylactic acid (PLA), polyethylene glycol (PEG), polycaprolactone (PCL), or their copolymers and combinations of these polymers)), polyplex, an antibody-drug conjugate, a liposome, an exosome, a virus like particle (VLP), a hydrogel, a microsphere/microcapsule (e.g., those made of polymers, including but not limited to, poly(lactic-co-glycolic) acid (PLGA), polyethlene glycol (PEG), polydimethylsiloxane (PDMS)), a suspension, a microvesicle, an implant (e.g., an implant for sustained release) or a combination thereof.

### Example 2

In aspects, an activator can be added to dBrec1—herein called BrecON. The activation of HIV transcription is part of a “Shock and Kill” treatment that has been proposed as potential approach to achieve HIV cure. This approach follows the rationale of activating non-active proviruses in reservoir cells (shock), in order for these cells to become susceptible to clearance by cytopathic effects or immune-mediated killing. BrecON can be used to achieve the activation of proviruses in this context (shock aspect).

BrecON relies on coupling an enzymatically inactive dBrec1 (see description above) to domains that promote transcriptional activation. These domains can be fused directly to dBrec1 or bind via one or multiple intermediate peptides. In the latter case, BrecOn consists of two or more components (example Suntag system). Recruitment of activator domains to the HIV LTR through dBrec1 support the establishment of open chromatin and a transcriptionally active environment. These include for example VP64 (4 copies of herpes virus transcriptional activation domain VP16), transcription factor p65 (nuclear factor NF-kappa-B p65 subunit) and heat shock factor 1 activator.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

Each and every patent, patent application, and publication, including publications listed herein and publicly available nucleic acid and amino acid sequences cited throughout the disclosure, is expressly incorporated herein by reference in its entirety. While this invention has been disclosed with reference to specific embodiments, it is apparent that other embodiments and variations of this invention are devised by others skilled in the art without departing from the true spirit and scope of the invention. The appended claims include such embodiments and equivalent variations.

What is claimed is:

1. A fusion protein comprising a DNA methyltransferase, a catalytically inactive Brec1 (dBrec1) and a Krüppel-associated box (KRAB).

2. The fusion protein of claim 1, wherein the DNA methyltransferase comprises a Dnmt3A protein.

3. The fusion protein of claim 2, wherein the Dnmt3A protein is linked to a Dnmt3L protein (Dnmt3A-3L protein).

4. The fusion protein of claim 1, comprising from N-terminus to C-terminus, a DNA methyltransferase, a catalytically inactive Brec (dBrec1), and a Krüppel-associated box (KRAB).

5. The fusion protein of claim 1, further comprising an epitope tag, a fluorescent protein tag, a nuclear localization signal peptide, or a combination thereof.

6. The fusion protein of claim 1, further comprising one or more linkers.

7. The fusion protein of claim 6, wherein the one or more linkers are XTEN linkers.

8. The fusion protein of claim 1, wherein dBrec1 is a mutated Brec1.

9. The fusion of protein of claim 8, wherein the mutation is at residue 323 of Brec.

10. The fusion protein of claim 9, wherein tyrosine at residue 323 in Brec is mutated to a phenylalanine.

11. The fusion protein of claim 1, wherein dBrec1 has the amino acid sequence of SEQ ID NO: 2 or 97% identity thereto.

12. A cell comprising the fusion protein of claim 1.

13. The cell of claim 12, wherein the cell is a eukaryotic cell.

14. The cell of claim 13, wherein the cell is a mammalian cell.

15. The cell of claim 13, wherein the cell is stem cell.

16. A method of silencing a HIV in a cell comprising delivering a polynucleotide sequence coding for the fusion protein of claim 1 to a cell containing HIV to silence HIV.

17. A method of treating HIV in a subject in need thereof comprising delivering to the subject an effective amount of a polynucleotide sequence coding for the fusion protein of claim 1 to treat HIV.

18. The method of claim 17, wherein the subject is human.

19. The method of claim 17, wherein the polynucleotide sequence is administered in a nanoparticle delivery vehicle.

20. The method of claim 17, wherein the polynucleotide sequence is administered virus like particle (VLP) delivery vehicle.


SEQUENCE LISTING (30 sequences)

SEQ ID NO: 1 - protein, 342 aa, synthetic construct
SILLTLHQSLSALLVDATSDEARKNLMDVLRDRQAFSERTWKVLLSVCRTWAAWCKLNNRKWFPAEPEDVRDYLLHLQARGLAVNTILQHLAQLNMLHRRFGLPRPGDSDAVSLVMRRIRRENVDAGERTKQALAFERTDFDQVRALMENSERGQDIRTLALLGVAYNTLLRVSEIARIRIKDISRTDGGRMLIHISRTKTLVSTAGVEKALSLGVTKLVERWISVSGVASDPNNYLFCQVRINGVAVPSATSRLSTDVLRKIFEAAHRLIYGAKDGSGQRYLAWSGHSARVGAARDMARAGVSIAEIMQAGGWTTVESVMNYIRNLDSETGAMVRLLEDGD

SEQ ID NO: 2 - protein, 342 aa, synthetic construct
SILLTLHQSLSALLVDATSDEARKNLMDVLRDRQAFSERTWKVLLSVCRTWAAWCKLNNRKWFPAEPEDVRDYLLHLQARGLAVNTILQHLAQLNMLHRRFGLPRPGDSDAVSLVMRRIRRENVDAGERTKQALAFERTDFDQVRALMENSERGQDIRTLALLGVAYNTLLRVSEIARIRIKDISRTDGGRMLIHISRTKTLVSTAGVEKALSLGVTKLVERWISVSGVASDPNNYLFCQVRINGVAVPSATSRLSTDVLRKIFEAAHRLIYGAKDGSGQRYLAWSGHSARVGAARDMARAGVSIAEIMQAGGWTTVESVMNFIRNLDSETGAMVRLLEDGD

SEQ ID NO: 3 - protein, 71 aa, Homo sapiens
DAKSLTAWSRTLVTFKDVFVDFTREEWKLLDTAQQIVYRNVMLENYKNLVSLGYQLTKPDVILRLEKGEEP

SEQ ID NO: 4 - protein, 301 aa, synthetic construct
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 5 - protein, 215 aa, synthetic construct
MGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 6 - protein, 543 aa, synthetic construct
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 7 - protein, 7 aa, synthetic construct
PKKKRKV

SEQ ID NO: 8 - protein, 7 aa, synthetic construct
GGSGGGS

SEQ ID NO: 9 - skipped

SEQ ID NO: 10 - protein, 17 aa, synthetic construct
EASGSGRASPGIPGSTR

SEQ ID NO: 11 - protein, 4 aa, synthetic construct
SRAD

SEQ ID NO: 12-13 - skipped (2 entries)

SEQ ID NO: 14 - protein, 26 aa, synthetic construct
SGNSNANSRGPSFSSGLVPLSLRGSH

SEQ ID NO: 15 - protein, 9 aa, synthetic construct
YPYDVPDYA

SEQ ID NO: 16 - protein, 19 aa, synthetic construct
ATNFSLLKQAGDVEENPGP

SEQ ID NO: 17 - protein, 7 aa, synthetic construct
GGSGGGS

SEQ ID NO: 18 - skipped

SEQ ID NO: 19 - protein, 17 aa, synthetic construct
EASGSGRASPGIPGSTR

SEQ ID NO: 20 - protein, 4 aa, synthetic construct
SRAD

SEQ ID NO: 21-22 - skipped (2 entries)

SEQ ID NO: 23 - protein, 9 aa, synthetic construct
YPYDVPDYA

SEQ ID NO: 24 - protein, 27 aa, synthetic construct
SSGNSNANSRGPSFSSGLVPLSLRGSH

SEQ ID NO: 25 - protein, 19 aa, synthetic construct
ATNFSLLKQAGDVEENPGP

SEQ ID NO: 26 - protein, 16 aa, synthetic construct
SGSETPGTSESATPES

SEQ ID NO: 27 - protein, 80 aa, synthetic construct
GGPSSGAPPPSGGSPAGSPTSTEEGTSESATPESGPGTSTEPSEGSAPGSPAGSPTSTEEGTSTEPSEGSAPGTSTEPSE

SEQ ID NO: 28 - protein, 232 aa, Aequorea victoria
SELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 29 - DNA, 34 bp, Human immunodeficiency virus 1
  source 1..34: mol_type = other DNA
aacccactgcttaagcctcaataaagcttgcctt

SEQ ID NO: 30 - DNA, 1023 bp, synthetic construct
  source 1..1023: mol_type = other DNA
atgtccatcctgctgaccctgcaccagagcctgtctgccctgctggtggatgccacctccgacgaggcccggaagaacctgatggacgtgctgagagacagacaggccttcagcgagcggacctggaaggtgctgctgagcgtgtgtagaacctgggccgcctggtgcaagctgaacaaccggaagtggttccccgccgagcctgaggatgtgcgggactacctgctgcatctgcaagccagaggcctggccgtgaacaccatcctgcagcatctggcccagctgaacatgctgcacagaagattcggcctgcccagacccggcgatagcgacgctgtgtctctcgtgatgcggcggatccggcgcgagaatgtggatgccggcgagagaacaaagcaggccctggccttcgagagaaccgacttcgatcaagtgcgggccctgatggaaaacagcgagagaggccaggacatccggaccctggctctgctgggcgtggcctacaataccctgctgcgggtgtccgagatcgcccggatcagaatcaaggacatcagccggaccgacggcggcagaatgctgatccacatctcccggaccaagaccctggtgtccaccgctggcgtggaaaaggccctgtctctgggcgtgaccaagctggtggaacggtggatctccgtgtctggcgtggccagcgaccccaacaactacctgttctgccaagtgcggatcaacggcgtggccgtgccttccgctacaagcagactgagcaccgacgtgctgcgcaagatcttcgaggccgcccacagactgatctacggcgccaaggatggcagcggccagagatacctggcttggagcggacacagcgccagagtgggagccgctagagatatggccagagccggcgtgtccattgccgagatcatgcaggctggcggctggaccacagtggaaagcgtgatgaactacatccgcaacctggacagcgaaaccggcgccatggtgcgcctgctggaagat